(19) United States
(12) Patent Application Publication
(10) Pub. No.: US 2025/0248624 A1
(43) Pub. Date: Aug. 7, 2025

(54) Non-Invasive Blood Glucose Prediction by Deduction Learning System

(71) Applicant: Academia Sinica, Taipei (TW)

(72) Inventors: Fu-Liang YANG, Hsinchu City (TW); Wei-Ru LU, Taipei (TW); Wen-Tse YANG, New Taipei City (TW); Tung-Han HSIEH, New Taipei City (TW); Justin CHU, Taoyuan City (TW)

(73) Assignee: Academia Sinica, Taipei (TW)

(21) Appl. No.: 18/854,965

(22) PCT Filed: Apr. 13, 2023
(86) PCT No.: PCT/US2023/018579

§ 371 Date: Oct. 7, 2024

Related U.S. Application Data
(60) Provisional application No. 63/331,234, filed on Apr. 14, 2022.

(51) Int. Cl. A61B 5/145; A61B 5/00; A61B 5/024; G16H 10/60

(52) U.S. Cl. CPC A61B 5/14532; A61B 5/02416; A61B 5/7246; G16H 10/60

(57) ABSTRACT

The present invention relates to a system and method for predicting blood glucose levels in a non-invasive manner using deduction learning. The deduction learning (DL) model 10 of the present invention predicts blood glucose level 300 based on the relationship or correlation between blood glucose level variation and PPG signal variation. Specifically, the DL model 10 of the present invention comprises a differential cell 100 (DC) configured to calculate predicted blood glucose level BGpred 300 using two PPG signals Si 200b and Si−1 200a, a BG i−1,ref 230 and relationship or correlation between variances of the two PPG signals and variances of the reference blood glucose level 230 and the predicted blood glucose level 300.

### FIELD OF THE INVENTION

The present invention relates to a system and method for predicting blood glucose levels in a non-invasive manner using deduction learning, allowing patients to receive care via personalized medicine and precision medicine.

### BACKGROUND OF THE INVENTION

Diabetes mellitus (DM) is a chronic condition of abnormally elevated blood glucose level (BGL) that typically leads to complications and damages to various parts of the body, and may further result in heart disease, kidney failure, blindness, and amputations. Blood glucose monitors are used to monitor and control the progression of DM and may be used to predict the risk of DM in a subject. However, most of the currently available glucose monitors utilize invasive methods requiring obtaining blood samples which causes pain and discomfort as well as exposes patients to risk of infectious diseases. In attempts to create systems for non-invasive blood glucose (NIBG) measurement, many have tried combining big data analysis and AI to provide blood glucose estimations. Taking into consideration physiological complexities and differences between individuals, there is a need for a personalized and precise blood glucose prediction system and method that utilizes non-invasive prediction methods.

Evidence suggests that photoplethysmography (PPG), an optical signal measurement technique based on near-infrared (NIR) transmittance or reflectance, may be a convenient and cost-effective choice for NIBG. PPG signal can be used to predict BGL by applying different morphologic feature extraction and signal processing techniques, such as Fast Fourier transform (FFT), wavelet transform, and Kaiser-Teager energy and spectral entropy. However, currently, there is no evidence of strong correlation between PPG optically-derived features with BGL, and large variability in human physiology leads to an enormous discrepancy of the extracted PPG-signal features with respect to BGL. Thus, personalized modeling by tracking BGL with different data collection methods has been explored to bridge the gap between the PPG signal and BGL, e.g. the works of Al-Dhaheri et al.¹ using PPG signals and a trained linear regression model, Rachim and Chung² using a linear partial least square regression (PLSR) model built from data of blood glucose pre-carbohydrate-rich meals and post-meals, and Yeh et al.³ using a linear least square regression model trained with blood glucose data after regular meals. Though they reported promising results, their prediction performance showed large variations from person to person, suggesting that either the models or the data collection methods may not be generally suitable for every subject. Furthermore, their prediction power over a long period remains unknown. ¹ Al-Dhaheri, M. A. et al. Noninvasive blood glucose monitoring system based on near-infrared method. International Journal of Electrical & Computer Engineering (2088-8708) 10 (2020).² Rachim, V. P. & Chung, W.-Y. Wearable-band type visible-near infrared optical biosensor for non-invasive blood glucose monitoring. Sensors and Actuators B: Chemical 286, 173-180, doi:https://doi.org/10.1016/j.snb.2019.01.121 (2019).³ Yeh, S.-j. et al. Monitoring Blood Glucose Changes in Cutaneous Tissue by Temperature-modulated Localized Reflectance Measurements. Clinical Chemistry 49, 924-934, doi:10.1373/49.6.924 (2003).

As the aforementioned personalized models collect data continuously from fasting to post-meal periods, the accompanying drastic physiological condition changes increase the level of complexity for modeling BGL based on PPG signals. Therefore, the present invention focuses on tracking fasting BGL, the preferred clinical index for the management of hyperglycemia in DM patients⁴, to reduce such interference. ⁴ Davies, M. J. et al. Management of Hyperglycemia in Type 2 Diabetes, 2018. A Consensus Report by the American Diabetes Association (ADA) and the European Association for the Study of Diabetes (EASD). Diabetes Care 41, 2669-2701, doi:10.2337/dci18-0033 (2018).

Moreover, the works of Al-Dhaheri et al., Rachim and Chung, as well as Yeh et al. implemented models with the structure of conventional concurrent input-output, hereafter referred to as “Induction Learning” (IL), which lacks the consideration of causal effect from preceding data. In principle, IL should predict well if a lot of well annotated data sets are available for model training. However, for personalized NIBG modeling, it is impractical to collect a large number of data sets, because the collection of reference BGL by means of finger-pricks for model training is quite uncomfortable and very user-unfriendly in daily usage. Conventional IL is used as a baseline for reference in the instant invention, while the innovative method of Deduction Learning (DL) is developed with the aim of significantly improving prediction accuracy with very few rounds of data for model training, at the same time adjusting for limited data for training the personal model and its unavoidable outlier predictions.

### SUMMARY OF THE INVENTION

The present invention relates to a non-invasive blood glucose prediction model that predicts blood glucose level of a subject based on deduction learning comprising a differential cell, a first input, a second input, a reference blood glucose level input and a predicted blood glucose level output wherein the first input is based upon a first cardiovascular data collected from the subject at a first round of data collection; the second input is based upon a second cardiovascular data collected from the subject at a second round of data collection; the reference blood glucose level input is collected from the subject at the first round of data collection; the differential cell is configured to calculate the predicted blood glucose level of the subject at the time of the second round of data collection based upon the first input, the second input, the reference blood glucose level and correlation between differences between the two inputs and differences between the reference blood glucose level input and the predicted blood glucose level; and the correlation is learned by the model using deduction learning.

The present invention also relates to a method of generating a predicted blood glucose for a subject using the model of claim 1 comprising the steps of collecting the first and the second cardiovascular data as well as the reference blood glucose level from the subject; creating the first and second input based upon the first and second cardiovascular data; inputting the first and second inputs into the model; inputting the reference blood glucose level into the model; and generating the predicted blood glucose level using the model based on the correlation between differences between the two inputs and differences between the reference blood glucose level input and the predicted blood glucose level.

The present invention further relates to a method of training the model of claim 1 using deduction learning comprising the steps of quantifying differences between a first historical input and a second historical input using the differential cell; quantifying differences between a first historical reference blood glucose level and the second historical reference blood glucose level using the differential cell; generating a predicted blood glucose level using the differential cell based on the correlation between the differences between the first historical input and the second historical input and differences between the second historical blood glucose level and the predicted blood glucose level; and adjusting the correlation to minimize absolute value of the difference in value between the second historical reference blood glucose level and the predicted blood glucose level.

### BRIEF DESCRIPTION OF THE FIGURES

FIG. 1 shows schematic diagrams of training of induction learning (IL) 8 and deduction learning (DL) 10 models. FIG. 1A illustrates training of IL model 8 and

FIG. 1B illustrates training of DL model 10. Each diamond block in FIG. 1A and triangular block in FIG. 1B represent a single personal model (NN) and a differential cell (DC) 100, respectively, which take PPG signals as input and predicted BG as output. Both NN and DC share similar CNN architecture (see FIG. 3). The PPG signals S₁ 200a, S₂ 200b, S₃ 200c, S₄ 200d are recorded in chronological order, with their corresponding reference glucose levels BG_{1,ref} 230a, BG_{2,ref} 230b, BG_{3,ref} 230c, BG_{4,ref} 230d. For FIG. 1B, when Sᵢ and Sᵢ₋₁ connect to the input of the DC (see FIG. 2), the loss between the reference BG_{i,ref} 230 and the output prediction BG_{i,pred} 300 will be minimized by the backpropagation of the model. FIG. 1C illustrates 1-channel input of signal segment convolves with a specific filter, that generates a simpler pattern of features (e.g., the reverse of the input signal). FIG. 1E depicts 2-channel input generating extra and non-uniform features beyond the original signals. FIG. 1D, FIG. 1F each illustrates one window of the overlapped output of 256 filters from the first CNN layer of IL model 8 and DL 10 model, respectively.

FIG. 2 shows an embodiment of the DL model 10 of the present invention comprising differential cell 100 (DC) of the present invention.

FIG. 3 shows architecture of the DC model 10 of the present invention. The input array contains concatenation of PPG morphological features 240 (6 elements) and a segment of PPG signal (400 elements), with dimension (406, 2) in pairing methods. Then it is followed by five CNN layers 110 (CNN 1˜CNN 5), each layer 110 consists of the same internal structure as shown in the upper-right corner of the flow chart. The input and the output dimensions of the extracted feature maps are listed as “in” and “out” for each CNN layer 110. After going through the five layers and flatten 130, the array is merged with the BGL of the preceding round BG_{i-1,ref} 230 (the red boxes), normalized with batch normalization, and goes through two fully connected layers (Dense) 126, 128 before the output, in which the number of neurons in each layer is presented in the right. For the non-pairing method (IL), the array of the preceded round in the input (the green box) and BG_{i-1,ref} (the red boxes) are not presented.

FIG. 4 illustrates Accumulated Blood Glucose Concentration in Subjects. Each of the values shown in the plot represents the mean glucose concentration (mg/dl) of one round of measurements. Samples of the same individual are vertically aligned in ascending chronological order. Red samples are the rounds of measurements after the 12th round.

FIG. 5 demonstrates the result of a signal segment passing through a specific CNN filter to illustrate a possible reason of superior performance in DL 10 over IL 8. FIG. 5A and FIG. 5B show a typical example (round 7 of subject 1) of the major difference in the first convolutional layer 110 between 1-channel and 2-channel inputs, with FIG. 5A showing one window of the overlapped output of 256 filters from the first CNN layer of IL, and FIG. 5B showing one window of the overlapped output of 256 filters from the first CNN layer 110 of DL 10. As shown in FIG. 5A, with simply 1-channel input that the vector convolves to a specific filter (filter length=3), the corresponding output generates a linear combination of three adjacent input vector elements for each output data point, which is seen as a reverse pattern in Example 1, shown in FIG. 6A. On the other hand, for 2-channel input vectors such as FIG. 5B, they convolve separately and are then superimposed together to form the output. This step creates more possible variation of operations to the original signal, including the one shown in FIG. 6B.

FIG. 6A shows 1-channel input of signal segment convolving with a specific filter, that generates a simpler pattern of features (e.g. the reverse of the input signal).

FIG. 6B shows 2-channel input generates extra and non-uniform features beyond the original signals.

FIG. 7A illustrates model training and model validation processes with the first stage of screening using the model quality screening module of the present invention. This example shows data of rounds 1 to 4 used for model training and validation, and data of round 5th as the testing set (see descriptions in FIG. 7A). The “leave-one-out” method successively leaves one of the rounds out of the training set during model building. As a result, four models M1˜M4 are trained and then validated by their corresponding left-out round of data. By examining all the validation results in Clarke Error Grid (CEG), one obtains a validation confidence score Sy, which is used for the first stage of screening for the quality of the resulting model. The threshold value x is determined empirically. The numbers shown in CEG represent the model numbers of M1˜M4. The repeated numeric legends are due to replicated measurements from the experiment in each round.

FIG. 7B illustrates the testing processes with the second stage of screening using the outlier screening module 430. When the first stage of screening is passed using the model quality screening module 410, the final model M5 was repeatedly trained N times with the training data of all the preceded rounds from 1˜4 with different random number seeds. Then the data of round 5 is tested by M5 to gather all the predictions for calculating the test spread score ST. If ST passes the threshold y (systematically determined by ROC curve, see FIG. 8, then the median value M (BGpred) of all the M5 predictions is used as the final prediction.

FIG. 8 illustrates the screening threshold and pass/reject performance relationship of ROC curve in FIG. 8A, and reject ratio of IL 8 and DL 10 models in FIG. 8B. Model training with rounds 8˜15 for all the test subjects were lump-summed here. The red dots indicate how the models perform when the threshold value of screening is set at 0.07.

FIG. 9 compares the accuracy scores (RA) of each method on all 30 subjects in each test round of Example 3. FIG. 9A shows the accuracy score distribution in a box plot, while FIG. 9B shows the mean accuracy scores in a line chart.

FIG. 10 illustrates a typical PPG waveform measured from the subjects. FIG. 10A illustrates the raw signal, consisting of the low frequency part and the high frequency part. FIG. 10B illustrates the low frequency part of PPG signal. FIG. 10C illustrates the high frequency part of PPG signal, in which the wave peaks are annotated by red circles, and the wave valleys are labeled by green boxes.

FIG. 11 illustrates pairing mechanism of one of the replicates in DL model of the present invention. FIG. 11A illustrates extraction of signal segments. A signal segment (window) of a round of data is cut from each valley of the PPG waveform backwardly up to 400 points (1.6 seconds). FIG. 11B illustrates three morphological features 240 extracted from a window 205 of signal waveform. FIG. 11C illustrates the data structure of one sample (labeled by (i,j)) of paired windows for model input. It consists of a paired data arrays from window j of round i and a randomly selected window j′ of the adjacent round i−1, together with a reference BGL BG_{i-1,ref} 230. Each of the paired data arrays contains a morphological feature vector 240 and a signal vector of the selected window. The sample (i,j) yields a BG_{(i,j),pred} 300 by model prediction.

FIG. 12 illustrates Clarke Error Grid (CEG) plots of model predictions: FIG. 12A depicts CEG of IL model 8. FIG. 12B illustrates CEG of DL 10 model. FIG. 12C illustrates CEG of DL+S model 20. The data points are grouped into three categories: green symbols are results of 4^{th} to 7^{th} rounds, blue symbols are results of 8^{th} to 11^{th} rounds, and red symbols are results of 12^{th} to 15^{th} rounds, respectively. The table below each figure lists the proportion of data points found in each zone of the CEG. For DL 10 and DL+S 20 models, all data points of rounds 12 to 15 are found in zone A and B.

FIG. 13 illustrates comparison of DL+S model 20 predictions for patients with and without insulin treatment at rounds 12 to 15.

FIG. 14 illustrates predictions of rounds 13˜15 (each paired with round 12) by models built with a dozen rounds of training data (rounds 1˜12) for IL model 8 shown in FIG. 14A, DL model 10 shown in FIG. 14B and DL+S 20 model shown in FIG. 14C.

FIG. 15 depicts an embodiment of the process flow for training an embodiment of the DL model of the present invention.

FIG. 16 illustrates features captured by 256 filters of the first layer of CNN of IL and DL models 10 for all of our recruited subjects.

FIG. 17 illustrates the learning curves of IL 8 in FIG. 17A and DL models 10 in FIG. 17B, in log-scale.

FIG. 18 illustrates CEG plots of BGL predictions for rounds 13˜15 by the personalized Random Forest model, trained with 6 morphological features data only of rounds 1˜12 in FIG. 18A, and both 6 morphological features and PPG signals data of rounds 1˜12 in FIG. 18B.

FIG. 19 summarizes and compares previous works of PPG-based NIBG with personalized models and the present invention.

FIG. 20 shows the distribution profile of 30 subjects from round 6 to round 15. Six to fifteen recurring rounds of PPG signal measurements and invasive fasting glucose recordings from 30 volunteers were taken. Several rounds of measurements were collected from these test subjects. Measurements of PPG signals and invasive glucose values BGRef 230 were taken using the TI AFE4490 Integrated Analog Front End and Roche Accu-check mobile, respectively.

FIG. 21 shows the performance of 30 subjects from round 4 to round 15, wherein the A-Zone ratio is the ratio of data points located in the zone A of CEG plot.

FIG. 22 summarizes required training time and GPU resources for training of the CNN architecture. The column “training rounds” means training from round 1 to the listed rounds.

FIG. 23 presents the overall performance of three models, IL 8, DL 10, and DL+S 20 in groups of rounds 4˜7, 8˜11, 12˜15, and the total (4˜15).

FIG. 24 presents the performance of models with 1st to 12th rounds as training and rounds 13, 14, 15 as testing (each paired with round 12). Also presented are preliminary test results of personalized Random Forest (RF) models 6. The A-Zone ratio is the ratio of data points located in the zone A of CEG plot, and RA, MAE, RMAE, and Rp are defined in Eqs. 4, 5, 6, and 7, respectively. Since each sample has its RA value, the average and standard derivation for each model were presented.

FIG. 25 shows basic information of recruited subjects, including gender, insulin treatment, use of drugs for DM treatment, smoking status, age, height, BMI, waist circumference, weight, and number of repeats. All test subjects were DM patients and eight took insulin treatment while others did not.

FIG. 26 shows the proposed pseudo code for Induction Learning.

FIG. 27 shows the proposed pseudo code for Deduction Learning

FIG. 28 shows the proposed pseudo code for Deduction Learning with screening.

### DETAILED DESCRIPTION OF THE INVENTION

The compositions of the present invention can comprise, consist of, or consist essentially of the essential elements and limitations of the invention described herein, as well as any of the additional or optional ingredients, components, or limitations described herein.

As used in the specification and claims, the singular form “a” “an” and “the” include plural references unless the context clearly dictates otherwise. For example, the term “a” cell includes a plurality of cells, including mixtures thereof.

“About” in the context of amount values refers to an average deviation of maximum ±20%, preferably ±10% or more preferably ±5% based on the indicated value. For example, an amount of about 30 mol % anionic lipid refers to 30 mol %±6 mol %, preferably 30 mol %±3 mol % or more preferably 30 mol %±1.5 mol % anionic lipid with respect to the total lipid/amphiphile molarity.

A “subject,” “individual” or “patient” is used interchangeably herein, which refers to a vertebrate, preferably a mammal, more preferably a human.

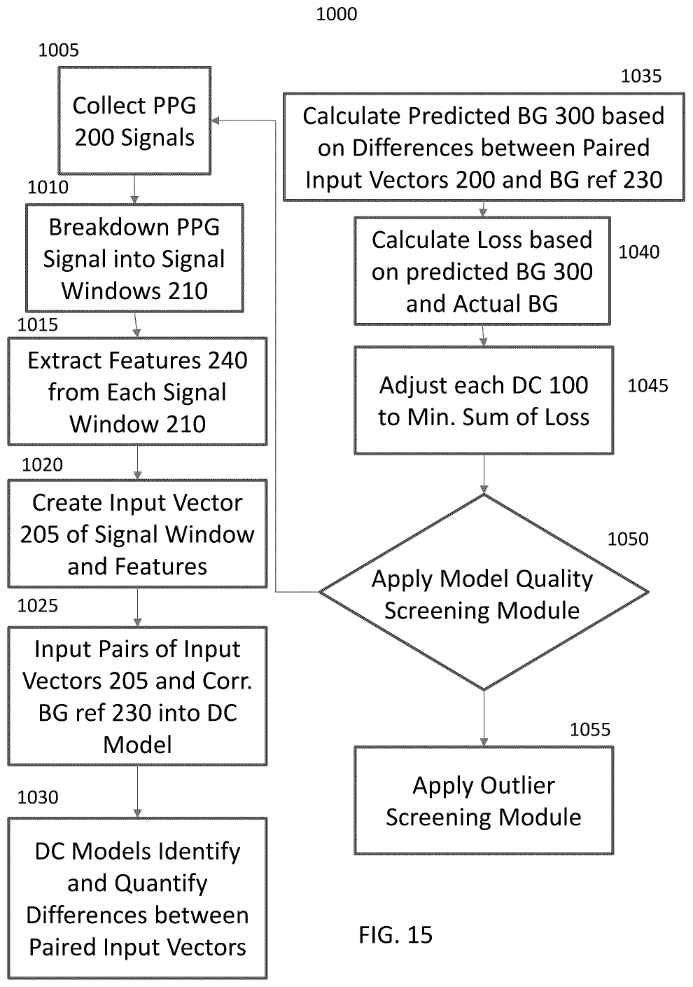

The deduction learning (DL) model 10 of the present invention predicts blood glucose level 300 based on relationship or correlation between blood glucose level variation and PPG signal variation. Specifically, if the difference of related physiological state change revealed in the differences of PPG signals Si 200b and Si−1 200a taken during two different rounds of data collection can be quantified and properly associated with blood glucose level change over the same time period, it is possible to encode and learn through pairs of adjacent PPG signals using deep neural network to predict blood glucose level even when association of the raw PPG signal and blood glucose level may be weak. Therefore, in an embodiment, the logic underlying the DL model 10 of the present invention comprises a relationship or correlation between change or differences in predicted blood glucose level from historical reference blood glucose level and change or differences in the corresponding PPG signals Si and Si−1 200a, 200b; such logic/relationship can be represented in equation form as:

$$D L ← {\sum }_{i = 2}^{N} f ( {S}_{i} , {S}_{i - 1} , B {G}_{i - 1 , ref} ) \qquad ( Eq . 1 a )$$

$$B {G}_{k , pred} ← D L ( {S}_{k} , {S}_{N} , {BG}_{N} ) \qquad ( Eq . 1 b )$$

where BGₖ,pred 300 is the predicted blood glucose level for a given measured PPG signal Sₖ 200 at round k of PPG data collection, BGᵢ,ref 230 (i=1˜N) are the historical or prior measured reference blood glucose level from a subject using conventional finger prick BG measurement, and Sᵢ 200 are the historical or prior measured PPG signal at round i (N<k), N is the total number of rounds of data for model training, and the function ƒ is a function to be deduced by machine learning that captures or comprises the relationship or correlation between PPG signal Si variances and corresponding blood glucose level variances. The index i indicates each round of PPG Si 200 and reference blood glucose BGi, ref 230 data collection wherein ascending numbers of i is ordered in chronological order so that adjacent i indicates successive rounds of Si 200 and BGi, ref 230 data collection. The DL model 10 of the present invention implements the deduction process of accumulated comparison between two consecutively measured PPG signals 200 that leads to successive corrections of the function ƒ by comparing the calculated BGpred 300 against the preceding ground truth BGᵢ₋₁,ref 230. Specifically, in an embodiment, the DL model 10 of the present invention implements the deduction learning process of accumulated comparisons of a plurality of BGi,pred 300 against corresponding plurality of BGi,ref 230, wherein each BGi,pred 300 is calculated by the model using Si−1 200a and Si 200b and BGi−1,ref 230, and adjusting the function ƒ to minimize cumulative difference in absolute value between BGi,pred and BG_{i,ref} in order to improve the function ƒ and, therefore, the predicted blood glucose level 300. The deduction learning process is discussed and illustrated in further detail below in connection with FIG. 15.

Therefore, the present invention provides a DL model 10 for predicting BG pred 300. In an embodiment, the DL model 10 of the present invention comprises a differential cell 100 (DC) configured to calculate predicted blood glucose level BGi,pred 300 using two PPG signals Si 200b and Si−1 200a, a BG i−1,ref 230 and relationship or correlation between variances of the two PPG signals and variances of the reference blood glucose level 230 and the predicted blood glucose level 300. In an embodiment, the two PPG signals Si 200b and Si−1 200a each comprises PPG signals from two different rounds of PPG signal collection i and i−1, and the BG i−1,ref 230 comprises reference blood glucose level acquired using conventional finger prick method obtained during the i−1 round of PPG signal collection.

In an embodiment, the PPG signals Si 200 and reference blood glucose levels 230 are collected from the subject in a fasting state. Fasting state of the subject 100 may be defined as the condition in which subject 100 has not had any intake of food and/or liquids for at least about 2 hours, at least about 3 hours, at least about 4 hours, at least about 6 hours or at least about 8 hours.

In an embodiment, the two consecutive rounds of PPG signal collection are separated in time by at least about 1 day to more than about 6 months such as about 1 day, about 2 days, about 5 days about 10 days, about 20 days, about 30 days, about 1.5 months, about 2 months, about 3 months, about 4, months, about 5 months, or about 6 months including all number ranges and numbers falling within these values.

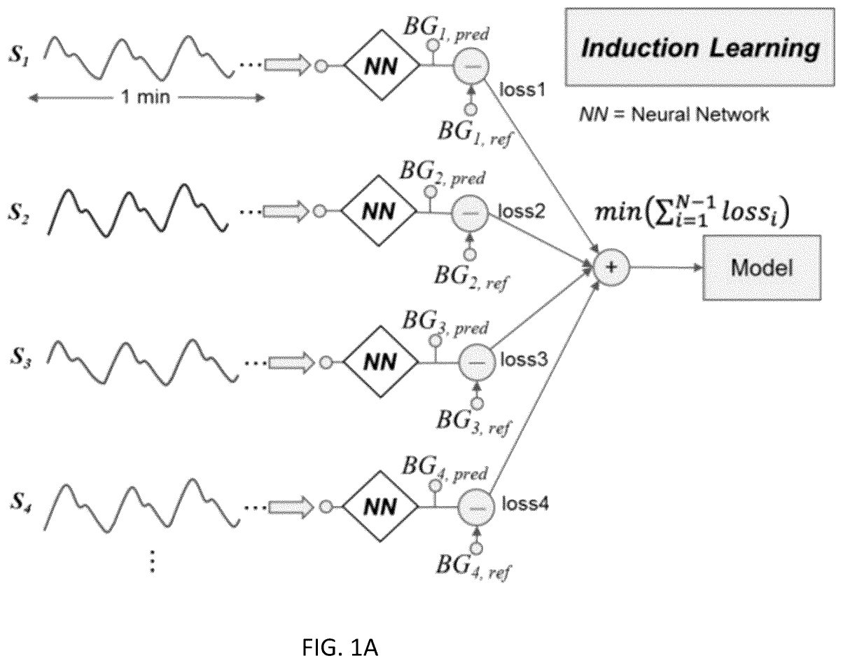

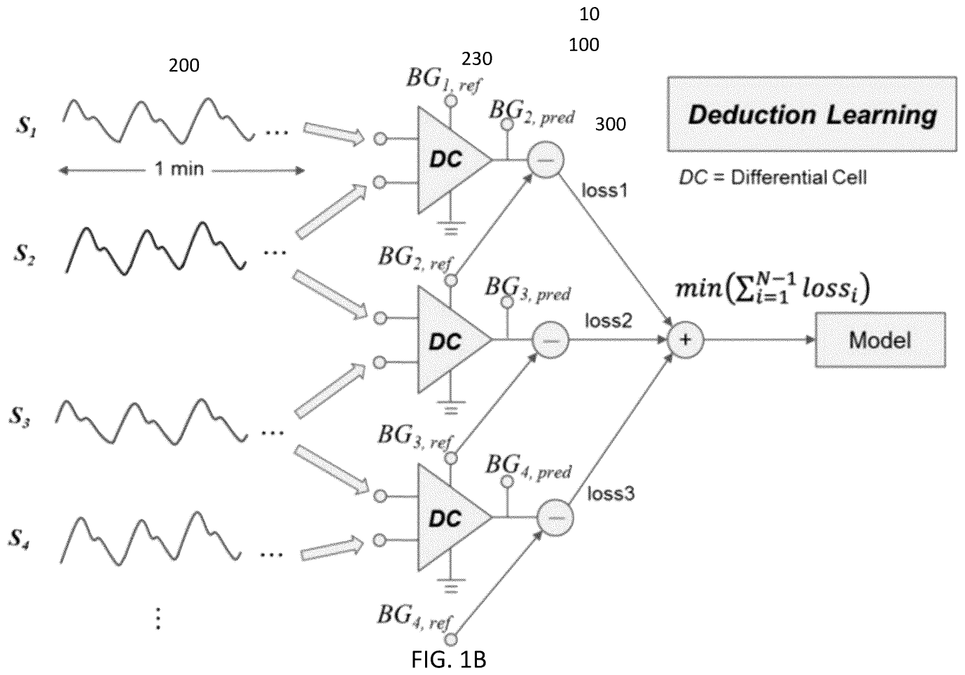

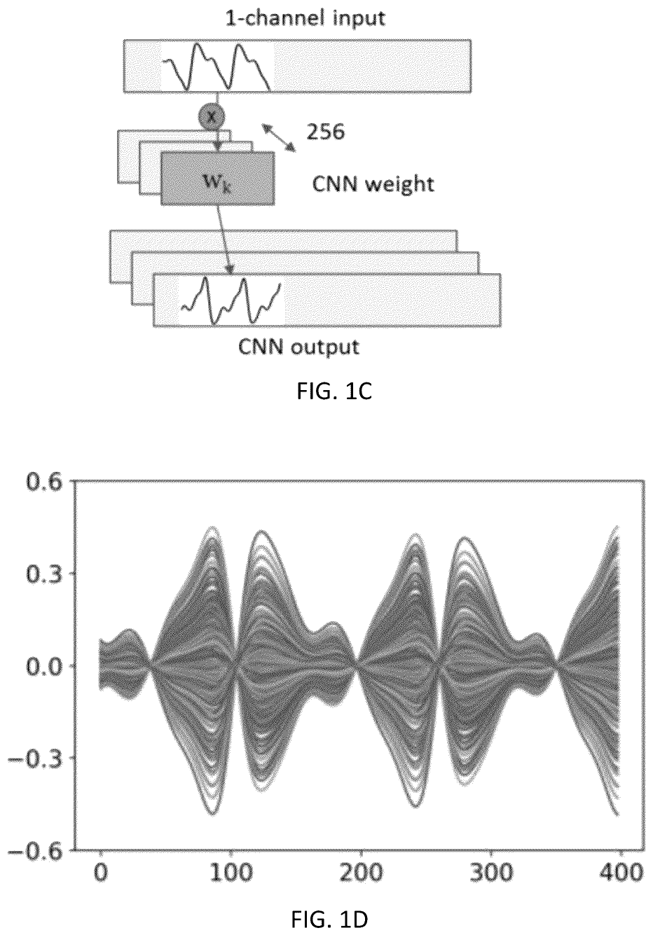

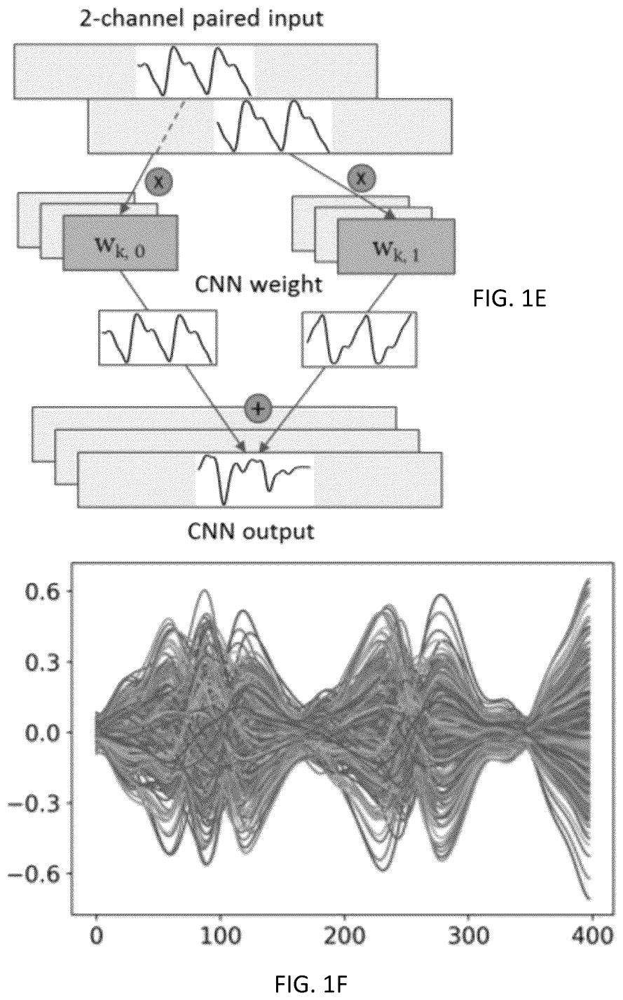

In an embodiment, the DL model 10 of the present invention is trained using a plurality of consecutive rounds of PPG signal collections such as more than 2 to more than 20 rounds of PPG signal collection such as about 2, about 3, about 4, about 5, about 6, about 7, about 8, about 9, about 10, about 12, about 14, about 16, about 18 or about 20 including any number ranges and numbers falling within these values. An exemplary DL 10 model training setup is illustrated in FIG. 1 and an exemplary DL training method is illustrated in FIG. 15 which will be discussed in further detail below. In an embodiment, the PPG signals 200 are input into the DL model 10 of the present Invention in the form of an input signal vector 205 wherein input signal vector 200 comprises a concatenation of digitized segments of the PPG signal called signal windows 210 with morphological features 240 extracted from the corresponding signal windows 210.

In an embodiment, the PPG signal 200 comprises PPG signals Sᵢ 200, S1200a, S2200b, S3200c, S4200d etc. which are acquired in chronological order in increasing value of index i along with corresponding reference glucose levels BG_{i,ref} 230, BG_{1,ref} 230a, BG_{2,ref} 230b, BG_{3,ref} 230c, BG_{4,ref} 230d wherein the index i indicates a particular round of PPG signal collection. In an embodiment, each Sᵢ 200 comprises PPG signal of a fixed time length of about 10 second to about 5 minutes such as about 10 seconds, about 30 seconds, about 1 minute, about 1.5 minutes, about 2 minutes, about 2.5 minutes, about 3 minutes, about 3.5 minutes about 4 minutes about 4.5 minutes or about 5 minutes including any numbers and number ranges falling within these values. An exemplary PPG signal 200 is illustrated in FIG. 10 (a).

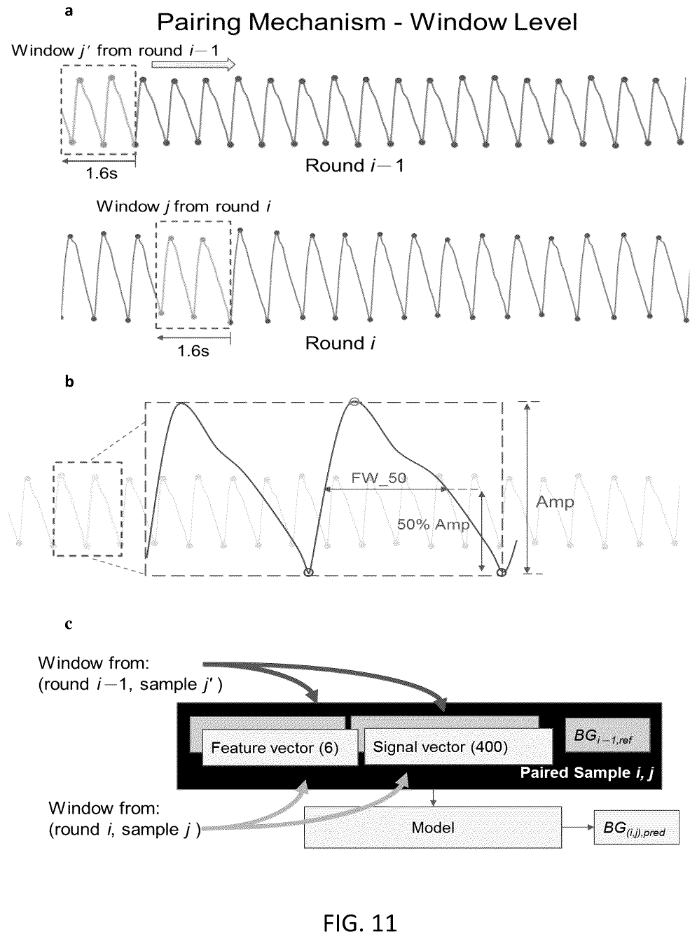

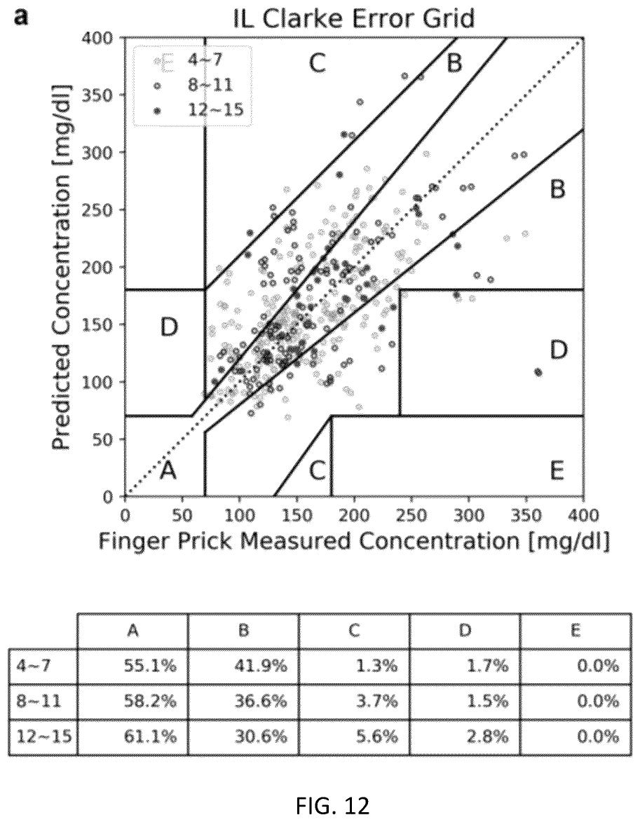

In an embodiment, each PPG signal Si 200 is digitized and broken up into a plurality of digitized signal windows 210 wherein each signal window 210 comprises or consists of a fixed time length of the PPG signal of about 1 second to about 20 seconds such as about 1 second, about 1.6 seconds about 2 seconds about 5 seconds about 10 seconds, about 15 second or about 20 seconds including any numbers and number ranges falling within these values. In an embodiment, each signal window 210 starts from each valley of the PPG signal 200 such that, if a subject's heart beats 60 beats/second, then a 1 minute long PPG signal 200 would result in 60 signal windows 210. In an embodiment, the signal windows 210 may be better defined using a low pass frequency filter as illustrated in the Examples below in connection with FIGS. 10 and 11. An example of a window of the present invention is illustrated in FIG. 11.

In an embodiment, one or more features 240 may be extracted from each signal window 210. In an embodiment, the features 240 comprises six morphological features including heart rate 242, area under the curve of the waveform 244, full width 246 and width at 25% 248a, 50% 248b 75% 248c of maximum peak amplitude (FW_25, FW_50, FW_75). Other features known in the art may also be included in the extracted features 240.

In an embodiment, an input to the DL model 10 of the present invention comprises one or more input signal vector 205 wherein each input signal vector 205 comprises a concatenation of the features 240 and one or more signal windows 210.

In an embodiment, the signal window 210 comprises a digitized segment of PPG signal 200 of 100 to 600 data points such as 100 data points, 200 data points, 300 data points, 400 data points, 500 data points or 600 data points including all numbers ranges and numbers falling within these values. In an embodiment, each input signal vector 205 comprises 400 PPG data points concatenated with six window features 240 to form an input signal vector 205 of 406 length.

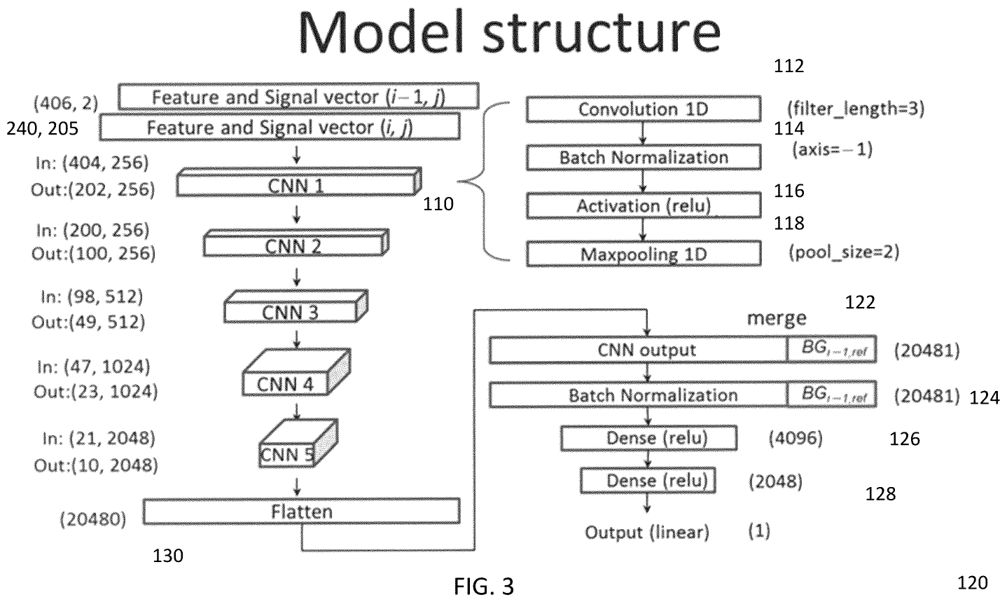

FIG. 3 illustrates an embodiment of a differential cell (DC) 100 of the present invention. As shown in FIG. 3, the DC 100 of the present invention comprises two inputs 101a and 101b each configured to receive a PPG signal input vector 205. The DC 100 of the present invention further comprises a reference blood glucose level input 231 for receiving reference blood glucose levels BG_{i,ref} 230.

As illustrated in FIG. 3, in an embodiment, each DC 100 of the present invention further comprises one or more convolution neural network (CNN) layers 110a, 110b, 100c . . . . In an embodiment, each CNN layer 110 is capable of performing 1D convolution. In an embodiment, each DC 100 comprises 1 to 10 CNN layers 110 such as 1 layer, 2 layers, 3 layers, 4 layers, 5 layers, 6 layers, 7 layers, 8 layers, 9 layers or 10 layers including all number ranges and numbers falling within these values. In an embodiment, each CNN layer 110 comprises a 1D convolution module 112. In an embodiment, the 1D convolution module 112 comprises filters 113 whose parameters may be adjusted during model training to improve the DC 100. In an embodiment, the 1D convolution module 112 comprises 256 to 2048 filters 113 whose parameters may be adjusted during model training. In an embodiment, the CNN layers 110 each further comprises a batch normalization module 114, a activation (relu) module 116 and/or a maxpooling module 118.

In an embodiment, the DC 100 of the present invention further comprises an output module 120 configured to receive BGref 230 as input and calculate BGpred 300 based on the result of CNN layers 110 and the BGref 230. In an embodiment, the DC output module 120 comprises a batch normalization module 124 and one or more Dense (relu (Rectified Linear Unit)) module 126. In an embodiment, the Dense relu module 126 comprises the function R(x)=x if x is larger than certain value, and R(x)=0 otherwise. In an embodiment, the DC 100 of the present invention further comprises a flattening module 130 in between the CNN layers 110 and the CNN output 122 configured to convert a multi-dimensional matrix to a one dimensional matrix. In an embodiment, the CNN output module 120 is configured to calculate BGpred 300 based on the result of CNN layers 110 and the BGref 230 using the batch normalization module 124 and the one or more Dense (relu) module 126. Each of the component of the DC 100 may comprise parameters that may be adjusted during model training to improve DC 100 blood glucose prediction. In an embodiment, DC 100 parameters such as filters 113 of each CNN layer 110, flatten 130 and dense parameters 126, 128 may be adjusted to modify the correlation between the variance of PPG signals 200 and the variance of reference blood glucose levels 230 and predicted blood glucose levels 300 in order to improve prediction accuracy of blood glucose. In an embodiment, the DC model 10 of the present invention including CNN layers 110 and other parts of the model may be constructed using various software packages such as Keras & TensorFlow, MXNET, Caffe, Torch & PyTorch etc. . . .

Although DL model 10 of the present invention was demonstrated to significantly outperform IL model 8 in prediction accuracy (see section Examples), a screening module 400 can be applied to further exclude outliers arising from abnormal measurement of PPG signals to result in the DL+S model 20 of the present invention. In an embodiment, the screening module 400 of the present invention comprises a model quality screening module 410. In another embodiment, the screening module further comprises an outlier screening module 430. In an embodiment, the model quality screening module 410 is configured to ensure trained DL model has been adequately and properly trained to calculate BGpred 300 using the Clark Error Grid (CEG) plot analysis. The outlier screening module 430 is configured ensure that BGpred outliers are eliminated using receiver operating characteristic (ROC) curve.

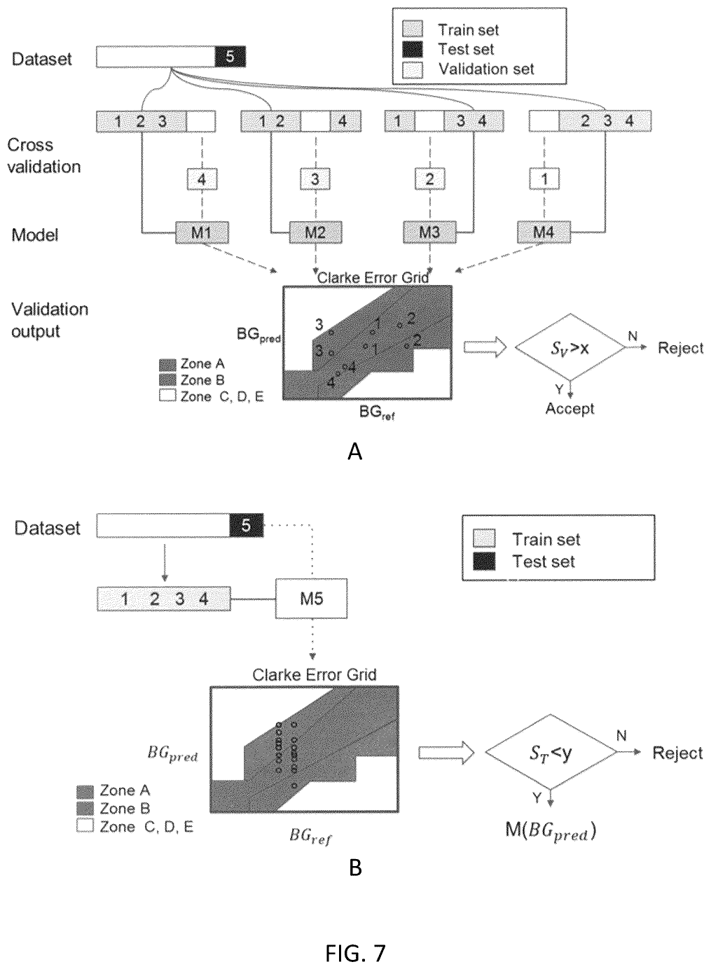

In an embodiment, the model quality screening module 410 of the present invention is based upon validation confidence score S_{V}. In an embodiment, the validation confidence score (S_{V}) is based on the scattered location of predicted BGs pred 300 and corresponding BGs ref 230 in Clark Error Grid (CEG) plot analysis as shown in FIGS. 7A and 7B constructed using the standard “leave-one-out” cross-validation procedure described in further detail in the Examples in connection with FIG. 7. By counting the accumulated validation data points inside each zone of the CEG plot, we define:

$${S}_{V} = {\sum }_{i ∈ U} {w}_{i} {C}_{i} / {\sum }_{i ∈ U} {C}_{i} , \qquad ( Eq . 2 )$$

where U stands for the union of all zones (from zone A to zone E), wₜ is the weight of zone i, and Cᵢ is the number of points inside zone i. When i=A, w_{A}=1; when i=B, w_{B}=0.5; and when i=C, D, and E, w_{C}=w_{D}=w_{E}=0. S_{V} is used to examine quality of the model built from the training data of all the preceded rounds. The model quality is highly sensitive to the amount and quality of the training data. If the model cannot pass the screening of the first stage, it usually means that more rounds of PPG measurements, together with the corresponding reference BGref from finger-prick measurements, are needed for model building.

In an embodiment, the outlier screening module 430 of the present invention is based upon the test spread score S_{T} parameter calculated from the model prediction of the training data and receiver operating characteristic (ROC) curve to filter out possible outliers. The outlier is usually caused by an inferior measurement of PPG signal, and redo the measurement more rigorously is usually necessary. In an embodiment, S_{T} is defined as the variation of the predicted BGC. “pred, i=1, 2, . . . N, by the N repeatedly trained models, with the maximum and minimum predicted values excluded:

$${S}_{T} = \sqrt{\frac{{\sum }_{i ∈ n} {( B {G}_{pred}^{( i )} - {\overline{BG}}_{pred} )}^{2}}{N - 1}} / Median ( {BG}_{p r e d} ) , \qquad ( Eq . 3 )$$

where set n={1, 2, . . . N} contains the repeatedly trained N rounds and |n|=N−2 due to removal of the maximum and minimum outcomes.

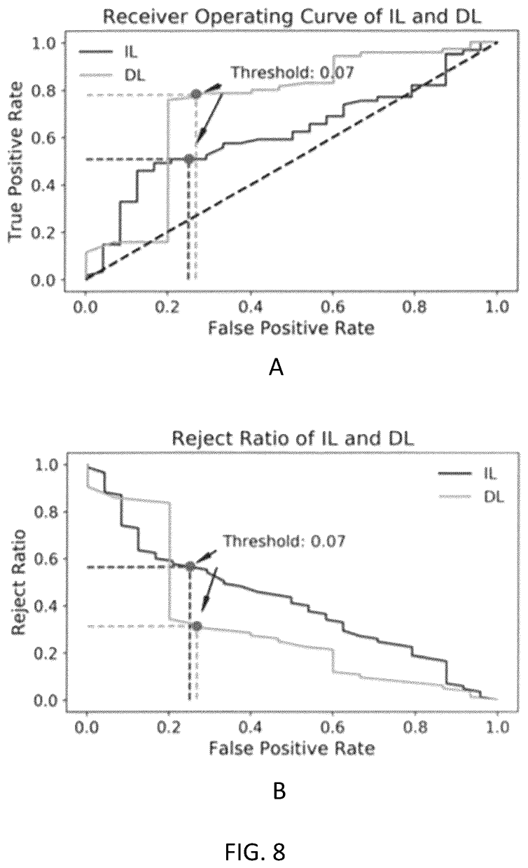

In an embodiment, ROC curve provides a systematic way to search for an optimal threshold value of S_{T} to filter out abnormal predictions. Ideally, the optimal threshold should be found near the convex point close to the upper-left corner of ROC curve plot. If the threshold is shifted along the curve to the upper right side, the condition is less stringent and more samples are accepted. If it is shifted towards the lower left side, the condition is stricter and fewer samples are included. FIG. 8 shows the ROC curve of the Example below calculated from all subjects after eight rounds of measurements for our models. It is interesting to compare the effect of S_{T} screening on both DL and IL, which reveals the superiority of DL over IL more clearly. The optimal threshold values of S_{T} for both cases are about 0.07, with the corresponding locations in ROC curve illustrated by the red dots. With this threshold value, the true positive rates of DL and IL are 78.3% and 50.8% (FIG. 8a), and the reject ratios are 31.1% and 56.5% (FIG. 8b), respectively. Furthermore, the ROC curve of IL is closer to the diagonal line, which indicates that the true positive rate and the false positive rate are simultaneously growing with respect to relaxing threshold. In this case, optimization of the threshold value would not improve the pass/reject accuracy significantly. On the contrary, the ROC curve of DL shows a promising shape above the diagonal line, which indicates the spread score S_{T} is more effective to distinguish the normal and abnormal test predictions in the DL model. Therefore, we conclude that pairing of adjacent rounds of data in DL helps screening task and achieves an overall better performance. In an embodiment, the threshold values of S_{T} may range from about 0.01 to 0.1 such as about 0.01, about 0.02, about 0.03, about 0.04 about 0.05, about 0.06, about 0.07, about 0.08, about 0.09 or about 0.1 including all numbers ranges and numbers falling within these values. In an embodiment, the outlier screening module is applied only after application of the model quality screening module.

The present invention also provides a method 1000 of training the DL model 10 of the present invention as illustrated in an exemplary DC model 10 training setup shown in FIG. 1 and the training method process shown in FIG. 15. As shown in FIG. 15, in step 1005, successive PPG signals Si 200 are collected from a subject at different rounds i wherein each round can be separated in time such as days, weeks or even months, and each PPG signal 200 is assigned the index i in chronological order.

So for example, S₁ is collected earlier than S₂ which is collected earlier than S₃, representing round 1, round 2 and round 3 etc. . . . in chronological order. In step 1010, the PPG signals 200 are broken down into individual digitized signal windows 210 as discussed above. Next, features 230 such as morphological features 240 are extracted from each window in step 1015. In step 1020, an input vector 205 is created for each signal window 210 by digitizing the section of the PPG signal 200 of the signal window 210 and concatenating the signal window 210 with the features 240. In step 1025, each input vector 205b is then paired with an input vector 205a from a preceding round of PPG signal collection. In an embodiment, the pairing can be with any random input vector 205 of PPG signal from a preceding round of PPG signal collection. As part of step 1025, the pair of input vectors 205a, 205b are then input into a DC 100 as shown in FIG. 1 along with BGi−1, ref 230 which is the BG measured by conventional finger prick method at the preceding round of PPG signal collection that serves as a reference BG. Therefore, if there are three DCs 100a, 100b and 100c, training would require a minimum of 4 PPG signals 200a, 200b, 200c and 200d and the corresponding 4 BGi−1, ref 230a, 230b, 230c and 230d.

In step 1035, each DC 100 identifies and quantifies changes or differences between each pair of input vectors 205a, 205b representing PPG data from two consecutive or adjacent PPG signals 200a, 200b and then calculates a predicted BGpred 300 based on the differences between the paired input vectors 205a, 205b and BG i−1,ref 230. In an embodiment, the identification and quantification of the differences is performed using a function ƒ configured to capture the relationship or correlation between changes or differences in the input vectors 205 and changes or differences of BGpred 300 and BG ref 230. In an embodiment, the prediction of blood glucose level is performed using deep neural network. In an embodiment, the prediction of blood glucose level is performed using convolution neural network. In step 1040, loss is the calculate as the absolute value of the difference between the BGi,pred 300 and the corresponding BGi, ref 230 for each DC 100 and total loss is calculated by totaling all the losses. The total loss is then minimized by the backpropagation of the DC model 10 in step 1045. In an embodiment, the adjustments to each DC 100 during training comprise weighting within the model structure illustrated in FIG. 3 including filters 113 of each CNN layer 110, flatten 130 and dense parameters 126, 128. In step 1050, model quality screening module 410 is applied to ensure trained DL model is adequately and properly trained to calculate BGpred 300 with adequate amount of accuracy. In step 1055, the outlier screening module 430 is applied to ensure that BGpred outliers are eliminated.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Materials and Methods

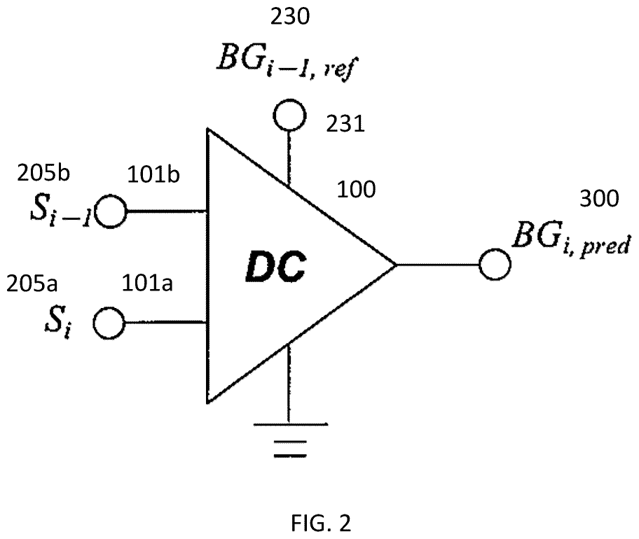

In this Example, our codes were developed in Python 3.6.6, with tensorflow 1.11.0, keras 2.2.4, on a platform of CUDA driver/runtime versions 10.1/9.0, and cuDNN 7.3.1.20. All the model training and testing were performed on an ASUS ESC8000 server with dual Intel Xeon Silver 4114 CPUs, 6 GPU cards of GTX 1080Ti (11 GB GPU on board memory), and 256 GB host memory. The training of the CNN architecture (FIGS. 2 and 3) was performed in GPUs, and the required training time and GPU resources are summarized in Table 4. The column “training rounds” means training from round 1 to the listed rounds. Note that our architecture of both IL and

DL are accumulated training, for example, training up to round 4 of DL involves three DC trainings of pairs (S₁,S₂), (S₂,S₃), and (S₃,S₄) (see FIG. 1). To speed up the trainings as much as possible, in each case we performed all the involved neural network NN (for IL) or DC (for DL) trainings parallelly. Thus, for trainings up to more rounds, the required GPU resources grew roughly linearly.

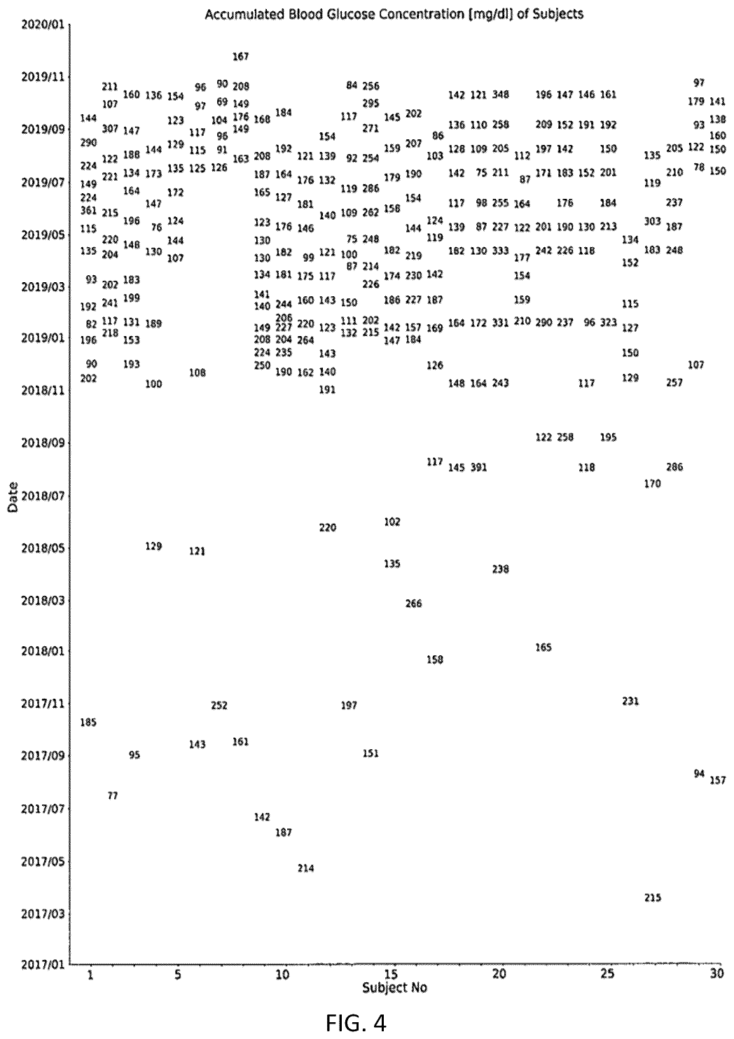

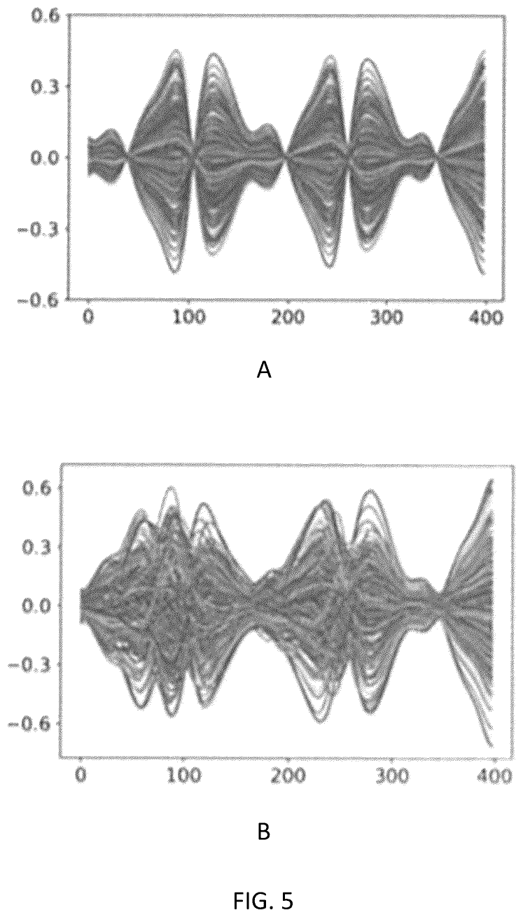

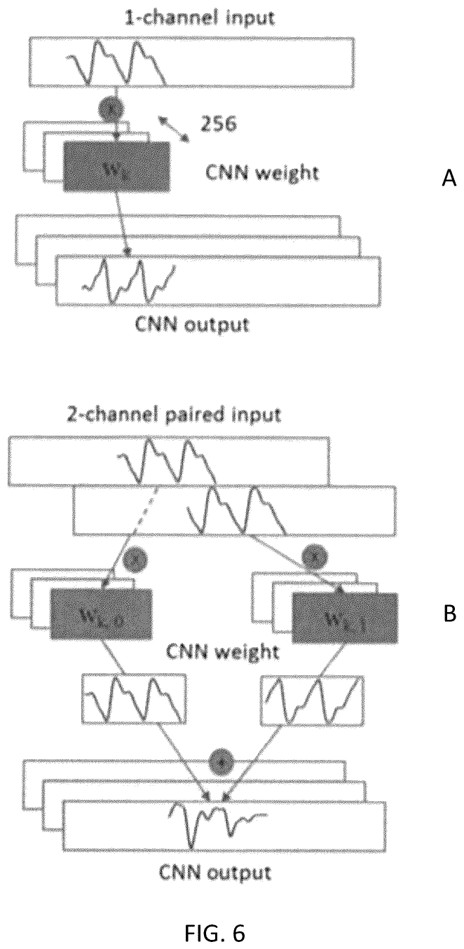

Sample Source. Six to fifteen recurring rounds of PPG signal measurements and invasive fasting glucose recordings from 30 volunteers were taken. All subjects were fully informed and written consents were obtained from all subjects for the collection of data and its uses. Since fasting BGL is relatively stable within a period of time, we set the duration of our sampling intervals to range from days to months in order to collect sufficient amounts of variations in BGL (FIG. 4). The collection of samples in this study has been approved by the Institutional Review Board of Academia Sinica, Taiwan (Application No: AS-IRB01-16081), and this study was performed in accordance with the relevant guidelines and regulations. Test subjects are all DM patients, of which eight took insulin treatment and others did not, as shown in Table 7. Several rounds of measurements were collected from these test subjects, as summarized in Table 2. Measurements of PPG signals and invasive glucose values were taken using the TI AFE4490 Integrated Analog Front End and Roche Accu-check mobile, respectively. Experiments were conducted inside the laboratory with a standard protocol including basic physical check-up, a questionnaire, and two replicates of 1-minute PPG signal measurements, each with a corresponding reference BGL measurement. The detailed experimental setup and procedures can be found in Reference³⁹.

Comparison of Models. The complete workflow of our DL+S model 20 contains PPG signal segmentation, feature extraction from PPG signal waveforms, data pairing, modeling, and screening, in which data pairing and screening are not presented in traditional IL model. To evaluate the importance of these key steps on the influence of model prediction power, the following three cases are compared.
1. Induction Learning (IL) 8: With one channel preprocessed signal as input of the CNN architecture, the accumulated data is trained with the traditional form [14-17]. Measurement data from the present round gives the prediction directly.
2. Deduction Learning (DL) 10: Pairing of adjacent rounds of measurements as a two-channel input, together with BGL measured in the preceding round of the paired input as the reference, to the CNN architecture.
3. Deduction Learning with screening enabled (DL+S) 20.
In all the models, the preprocessing of data of each round, the CNN architecture, and the procedures of model training, validation, and testing are all the same, as described detailly in the following.

Data Preprocessing. A typical PPG waveform 200 measured from the subjects is illustrated in FIG. 10. The raw signal reveals pulses in varied amplitudes (FIG. 10a), each pulse corresponds to a single heartbeat. The raw signal can be separated into low frequency part (FIG. 10b) and high frequency part (FIG. 10c) by a Butterworth filter⁴⁰ with the cutoff frequency of about 0.75 Hz. The high frequency part is used for the following feature extraction and model input. The valleys and peaks of the high frequency part is automatically annotated by Bigger-Fall-Side algorithm.⁴¹ The idea comes from observation of the pulses' waveform, i.e., every peak is immediately followed by a valley with the largest magnitude difference in the pulse, which we called the “bigger-fall-side-slope” (BFSS). Since most people have 60-90 heartbeats within one minute, we sorted all the available slopes between nearby local minimum and local maximum in descending order, and selected the 30th one as the medium of BFSS values (mBFSS). All the slopes fallen into the range [0.5, 1.5] of mBFSS were identified as BFSS. Thus, the peaks and valleys of the whole waveform can be correctly labeled.

By annotating the valleys of the waveform, a PPG signal segment which we called a window 210 is extracted from each valley backwardly to 400 data points earlier, which covers 1.6 seconds that include at least one pulse of the PPG waveform (FIG. 11a). For a subject with heart rate of 60 beats/min, 60 windows 210 are generated. Six morphological features 240 including heart rate 242, area under the curve 244 of the waveform, full width 246 and width at 25% 248a, 50% 248b, 75% 248c of maximum peak amplitude (FW_25, FW_50, FW_75) are extracted from the corresponding window (FIG. 11b). The extracted features 240 are then concatenated with the signal window 210 to form an input vector 205 with 406 elements. Thus, a round of measurement with two replicates generates data arrays with size (Nᵢ₁, 406) and (Nᵢ₂, 406), respectively, where Nᵢ₁ and Ndi2 represents the number of windows of the two replicates in the round i.

The following paragraph highlights differences between IL 8 and DL (+S) 10, 20 models. In preparing a sample input data of the replicate k (k=1,2) in round i to the CNN architecture, data of window jₖ (which is a vector containing 406 elements) is sampled out to form a 1-channel input for IL 8; while for DL (+S) 10, 20, the window jₖ in replicate k of round iₜₕ is paired with a randomly selected window j′ₖ from the same replicate k of the preceded round i−1 to form an input sample labeled (i,jₖ) (FIG. 11c). In our work, pairing involves the combination of samples between two consecutive rounds of measurement, which may be in a time span of days or months, depending on our accumulation of data from subjects. For both IL 8 and DL (+S) 10, 20, the total number of input samples of round i is Nᵢ₁ and Nᵢ₂ for the two replicates, because of the Nᵢ₁ and Nᵢ₂ windows available in two replicates of measurement in this round.

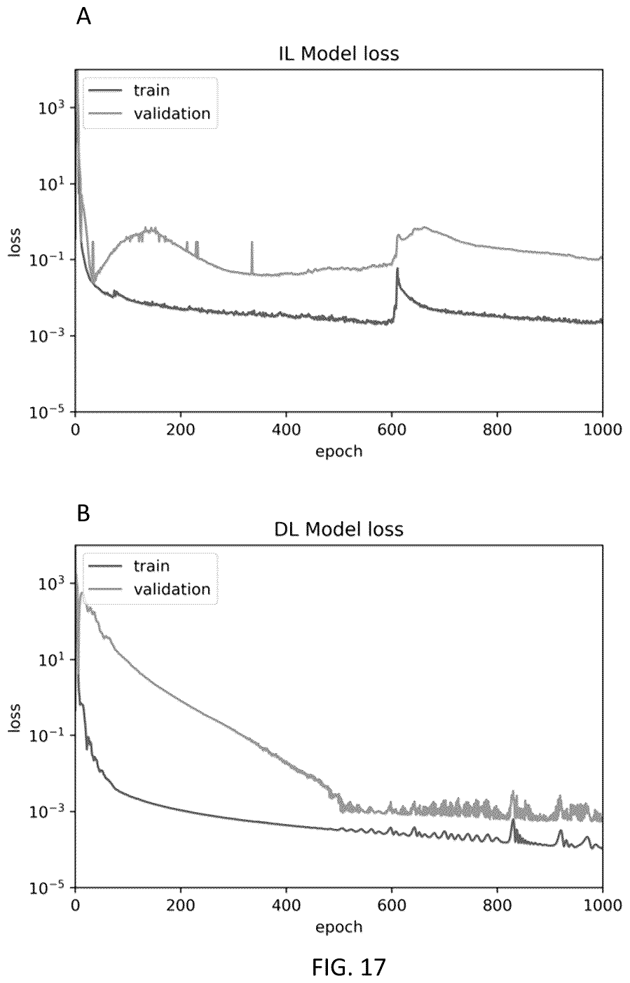

The Model. The detailed layout of our CNN architecture is illustrated in FIG. 3. For DL (+S) 10, 20, the samples with paired windows are designed as two channels for model input, each channel corresponds to one of the paired windows. The input data is then passed through five units of CNN layers 110 with number of filters 256, 512, 1024, and 2048. Our tests show that more CNN layers 110 may potentially give higher predicting accuracy. Since each CNN layer 110 consists of a maxpooling layer 118 with pool size 2, the data vector 205 is reduced to half when passed through each layer 110, restricting the number of CNN layers 110. The choice of number of filters in each CNN layer is a balance of extracting as many features 240 as possible from the input data while keeping the total amount of trainable parameters in the model under reasonable control. With our setting of number of filters, the total amount of trainable parameters is about 100,800,000, which is manageable in our GPU computing platform. The learning curves up to 1000 training epochs of our models (IL and DL) are presented in FIG. 17, which shows no overfitting since the curves of testing loss decreased together with the training loss. Due to the limit of the GPU memory, the batch size is set to 3000. Finally, Rectified linear units (ReLU), Adaptive Moment Estimation (Adam), and mean squared error were adopted as our activation function, optimizer, and loss function in our model, following common practice of CNN development. After the five layers of CNN 110, the flattened output is merged with BGL of the preceded round BGᵢ_{ₖ}_{-1,}ref (for replicate k), and then go to two fully connected layers after batch normalization to get the BGL prediction BGᵢ_{ₖ}_{,pred}. On the other hand, IL shares the same architecture except that there is only one channel for model input, as information of the preceding round is not considered.

Training, Validation, Testing, and Screening. The model training, validation, and testing processes rely on a well-defined data splitting configuration.

FIGS. 7A and 7B illustrate an example of taking data of round 5 as a prediction test. For the training and validation processes shown in FIG. 7A, the standard “leave-one-out”cross-validation procedure was performed on data of all the preceded 1^{st}˜4^{th} rounds to examine whether the model is overfitted. In addition, for the DL+S model 200 in which screening is enabled, we define a validation confidence score (S_{V}) based on the scattered location of predicted BGLs in CEG plot analysis. By counting the accumulated validation data points inside each zone of the CEG plot, we define:

$${S}_{V} = {\sum }_{i ∈ U} {w}_{i} {C}_{i} / {\sum }_{i ∈ U} {C}_{i} , \qquad ( Eq . 2 )$$

where U stands for the union of all zones (from zone A to zone E), wᵢ is the weight of zone i, and Cᵢ is the number of points inside zone i. When i=A, w_{A}=1; when i=B, w_{B}=0.5; and when i=C, D, and E, w_{C}=w_{D}=w_{E}=0. In other words, only data points in zone A and zone B receive non-zero weights, because these predictions are relatively acceptable BGL measurements clinically. During validation, the more predicted data found in zones A and B, the higher S_{V}. After calculating S_{V} from all the cross-validation data, total S_{V} was evaluated. If this score is lower than a pre-determined validation threshold, the process stops and rejects this model training as well as the following test prediction. The threshold value depends on the number of accumulated rounds of data, which is empirically set to 50 if test round is <7 and 60 if test round is >7. This is the first stage of the whole screening process, which is used to examine the quality of the trained model based on data of all the past rounds.

After the task of cross-validation, all the preceding data (i.e., data of rounds 1˜4 in this example) were used to train the final model, and the present data (i.e., data of round 5 in this example) was used to test the model, as shown in FIG. 7B. For DL+S 20, the final model was repeatedly trained N times, each with the same set of training data, but with different random number sequences, and tested by the same testing data. Thus, an additional screening procedure was carried out by the test spread score S_{T}, which is defined as the variation of the predicted BGLs BG_{pred}⁽ⁱ⁾, i=1, 2, . . . N, by the N repeatedly trained models, with the maximum and minimum predicted values excluded:

$${S}_{T} = \sqrt{\frac{{\sum }_{i ∈ n} {( B {G}_{pred}^{( i )} - {\overline{BG}}_{pred} )}^{2}}{N - 1}} / Median ( {BG}_{p r e d} ) , \qquad ( Eq . 3 )$$

where set n={1, 2, . . . N} contains the repeatedly trained N rounds and |n|=N−2 due to removal of the maximum and minimum outcomes. To do the screening, S_{T} must be smaller than a threshold value y, then the median value of all the BG_{pred}⁽ⁱ⁾ is accepted as the final prediction. This is the second stage of the whole screening process, which is to remove the abnormal outliers of the prediction. The optimal value of the threshold y was systematically determined by the ROC curve³⁶. In FIG. 8, the true/false positive rate of model predictions was investigated with respect to tuning of the threshold value y, and the optimal value should be near the convex point close to the upper-left corner of the plot of ROC curve. Since there are two replicates of measurement in each round, “true” is defined by either both predictions are in zone A, or one is in zone A while the other one is in zone B, of the CEG plot.

Performance Evaluation. The performance metrics of glucose prediction in test samples are calculated by the accuracy score (R_{A}), mean absolute error (MAE), root mean squared error (RMSE), and the Pearson correlation coefficient (Rₚ). They are defined as follows:

$${R}_{A , i} = \vert \frac{B {G}_{pred , i} - {BG}_{ref , i}}{B {G}_{ref , i}} - 1 \vert \times 100 \qquad ( Eq . 4 )$$

$$MAE = \frac{1}{n} {\sum }_{i} \vert {BG}_{p r e d , i} - B {G}_{r e f , i} \vert \qquad ( Eq . 5 )$$

$$RMS E = \sqrt{{\sum }_{i} {( B {G}_{p red , i} - B {G}_{r e f , i} )}^{2} / n} \qquad ( Eq . 6 )$$

$${R}_{p} = \frac{{\sum }_{i} ( B {G}_{ref , i} - \overline{{BG}_{ref}} ) ( {BG}_{pred , i} - \overline{{BG}_{pred}} )}{\sqrt{{\sum }_{i} {BG}_{ref , i}^{2} - {( {\sum }_{i} B {G}_{ref , i} )}^{2}} \sqrt{{\sum }_{i} {BG}_{pred , i}^{2} - {( {\sum }_{i} B {G}_{pred , i} )}^{2}}} \qquad ( Eq . 7 )$$

where i is the index of each samples, with i=1, 2, . . . , n.

### Model Design

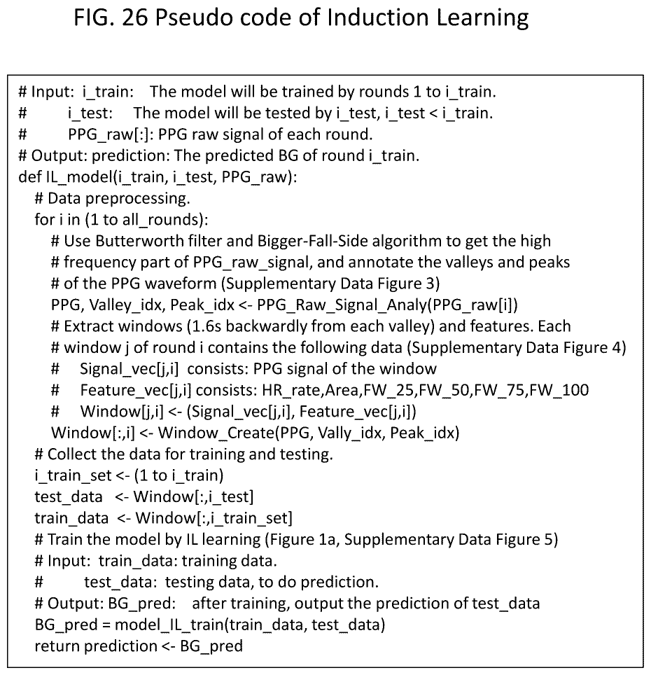

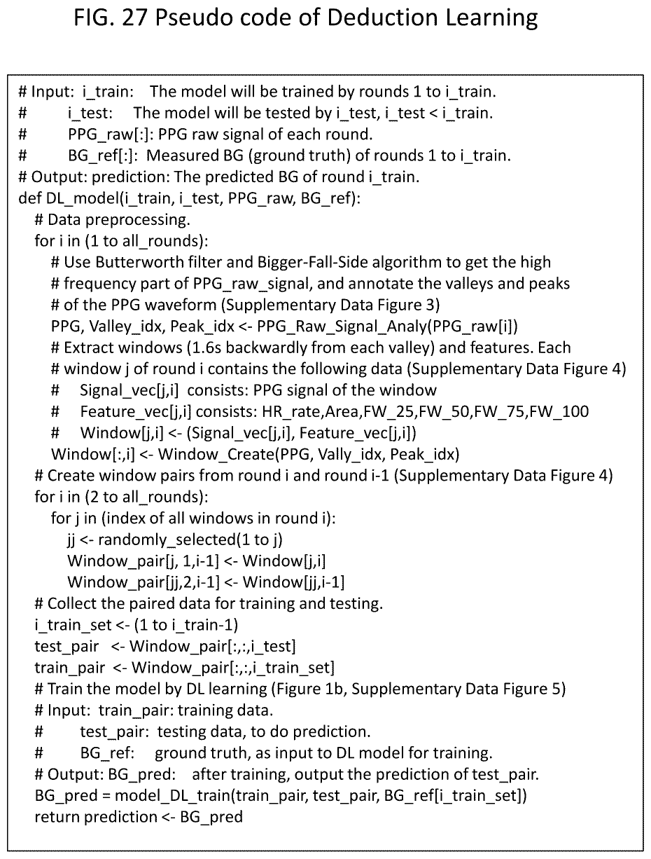

The schematic diagrams and pseudo codes of IL and DL models 8, 10 are presented in FIGS. 1a and 1b, and FIGS. 26-28, respectively. In this work, both IL and DL models 8, 10 were designed as the process of accumulated learning, with adding more and more rounds of training data. The rule imposed into our DL model 10 is the assumption of the relation between the predicted BGL 300 with its preceding BGL 230, and also the corresponding PPG signals 200 measured:

$$D L ← {\sum }_{i = 2}^{N} f ( {S}_{i} , {S}_{i - 1} , B {G}_{i - 1} ) \qquad ( Eq . 1 a )$$

$$B {G}_{k} ← D L ( {S}_{k} , {S}_{N} , {BG}_{N} ) \qquad ( Eq . 1 b )$$

where BGₖ 300 is the predicted BGL for a given measured PPG signal Sₖ 200 at round k, BGᵢ 300 and Sᵢ 200 are the preceding measured BGL (i=1˜N) for model training and PPG signal at round i (N<k), N is the total number of rounds of data for model training, and the function ƒ is unknown to be deduced by machine learning. In this work, we tentatively set N=12 for clinically acceptable predicting accuracy (see below). Plainly speaking, we aimed to implement the deduction process of accumulated comparison between two consecutively measured PPG signals 200 that leads to successive corrections to the preceding ground truth BGᵢ₋₁ 230.

In this study, personalized data (i.e., PPG and BGL) of the recruited subjects were collected in several rounds of measurements (FIG. 4) for models building and testing. Our DL model 10 of pairing input signals for BGL prediction was inspired by the scheme of a differential amplifier (DA). For a DA, it takes two signals (i.e., Sᵢₙ₊ and Sᵢₙ₋) as input, reduces the potential background noise, and amplifies the difference of the two input signals for output (i.e., Vₒᵤₜ) within a voltage range given by the source (i.e., V_{S+}). Analogically, our DL model 10 comprises successive sets of differential cells (DC) 100 (FIG. 2), each takes two signals Sᵢ 200b and Sᵢ-1200a (i.e., the adjacent rounds of PPG data) forming a pair of input and a reference BGL 230 (i.e., the precedingly measured BG_{i-1,ref}) as the baseline, and outputs the predicted BGL BGi,pred 300 based on the correlation between Sᵢ 200b and Sᵢ₋₁ 200a variations and BGi,pred 300 and BG_{i-1,ref} 230 variations. If the difference of related physiological state change revealed in the difference of Sᵢ 200b and Sᵢ₋₁ 200a can be quantified and associated with change in corresponding blood glucose levels, it is possible to encode and learn through pairs of recordings by a deep neural network, even though the association between the raw signal and glucose concentration may be weak.

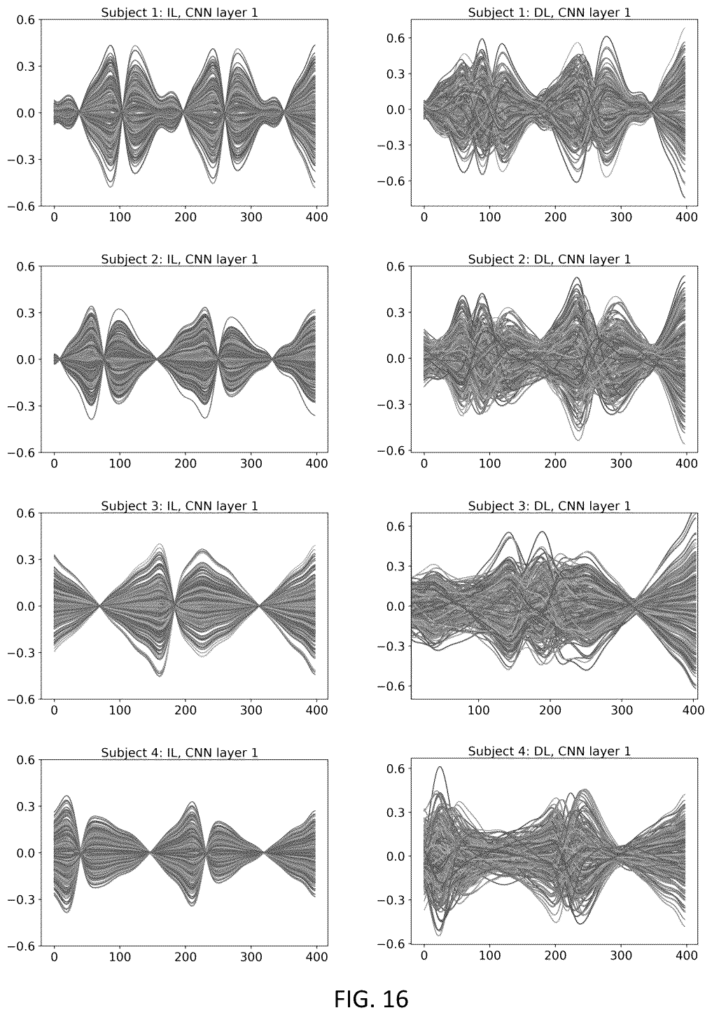

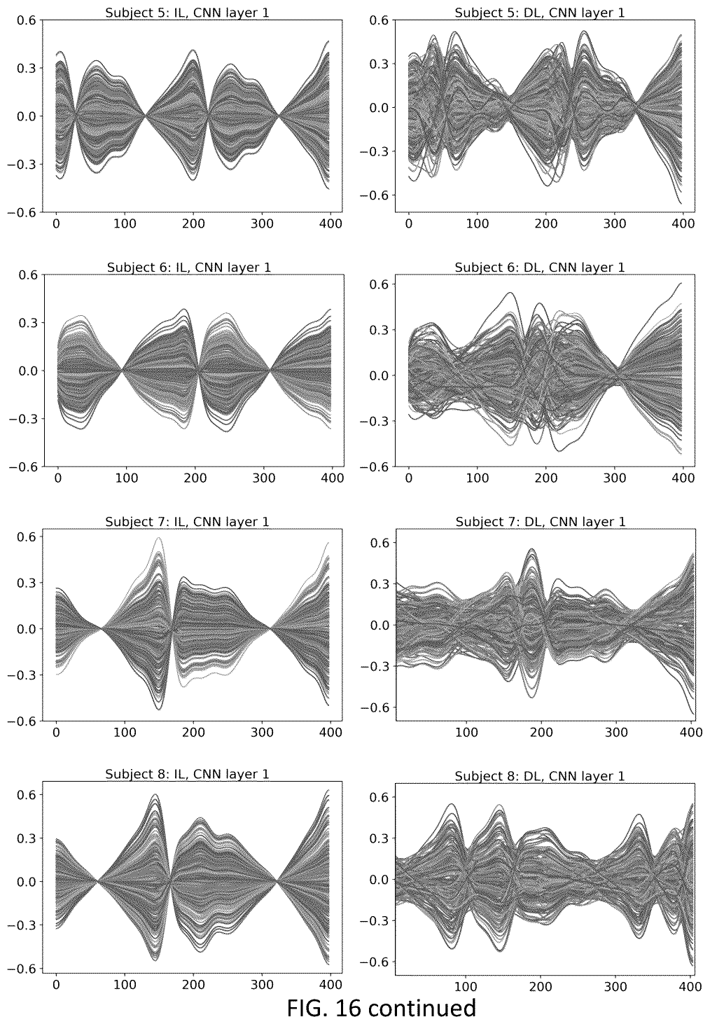

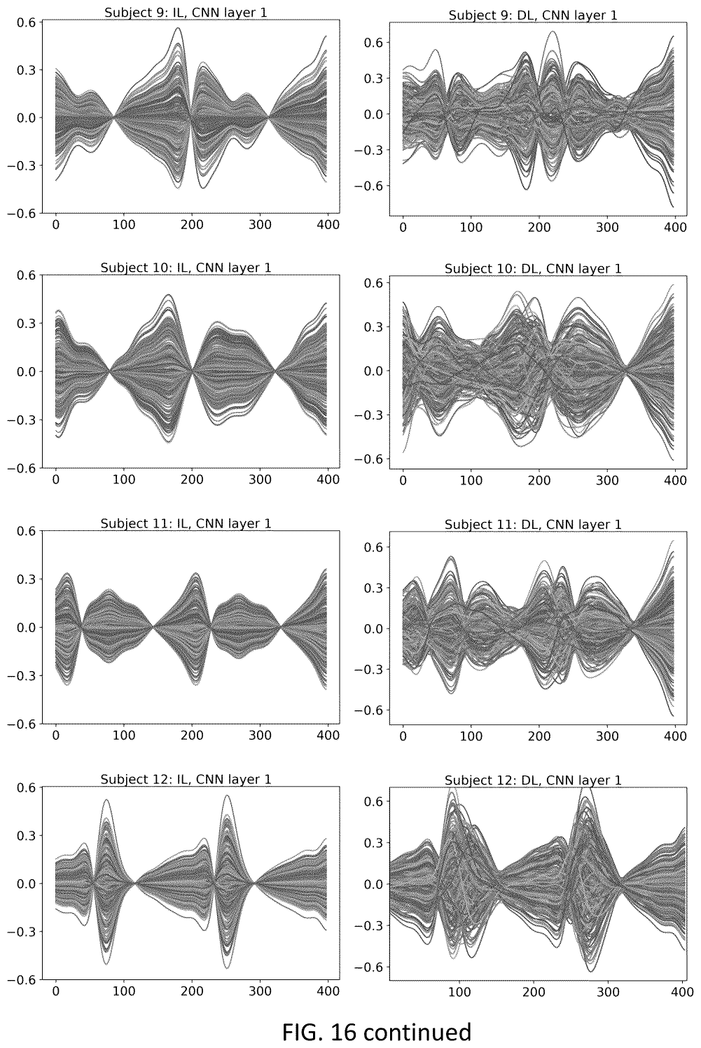

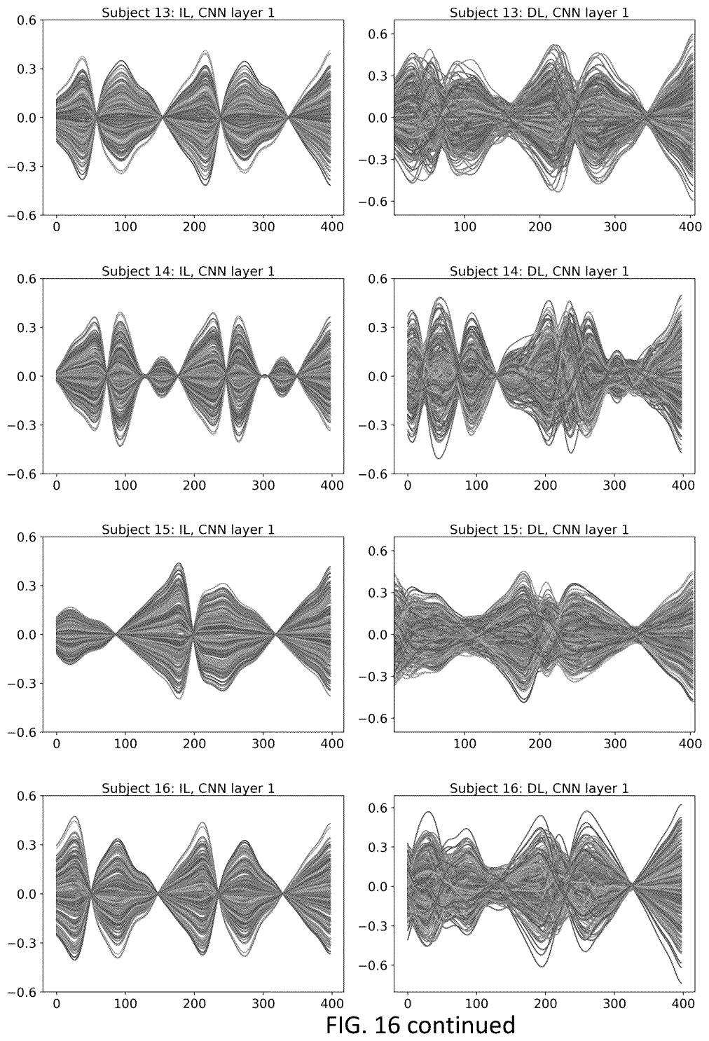

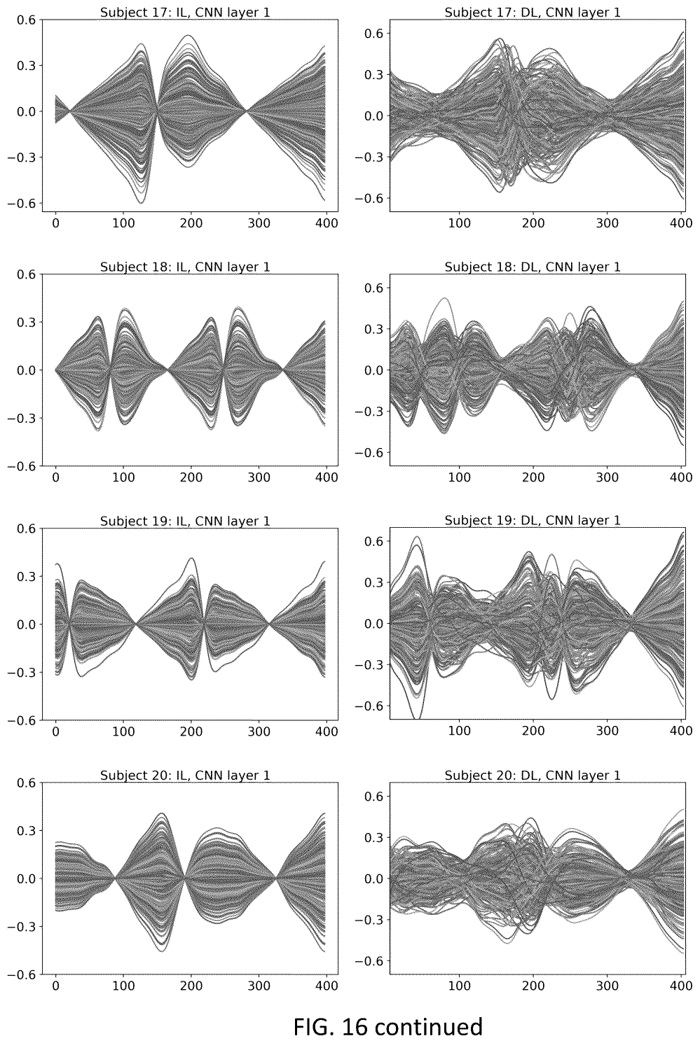

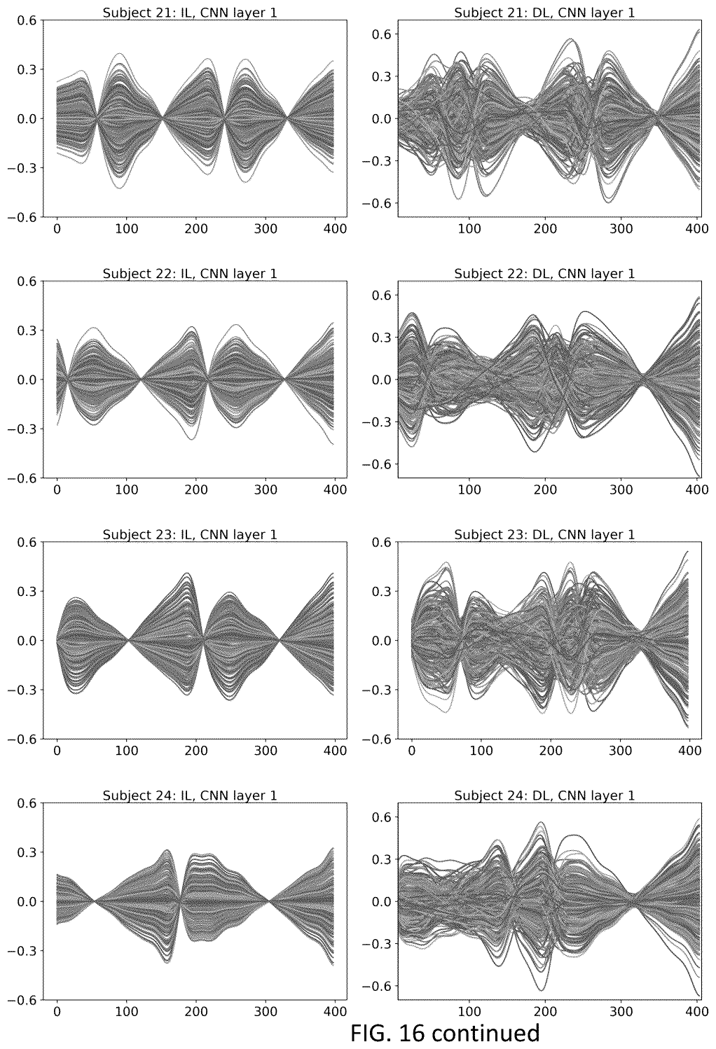

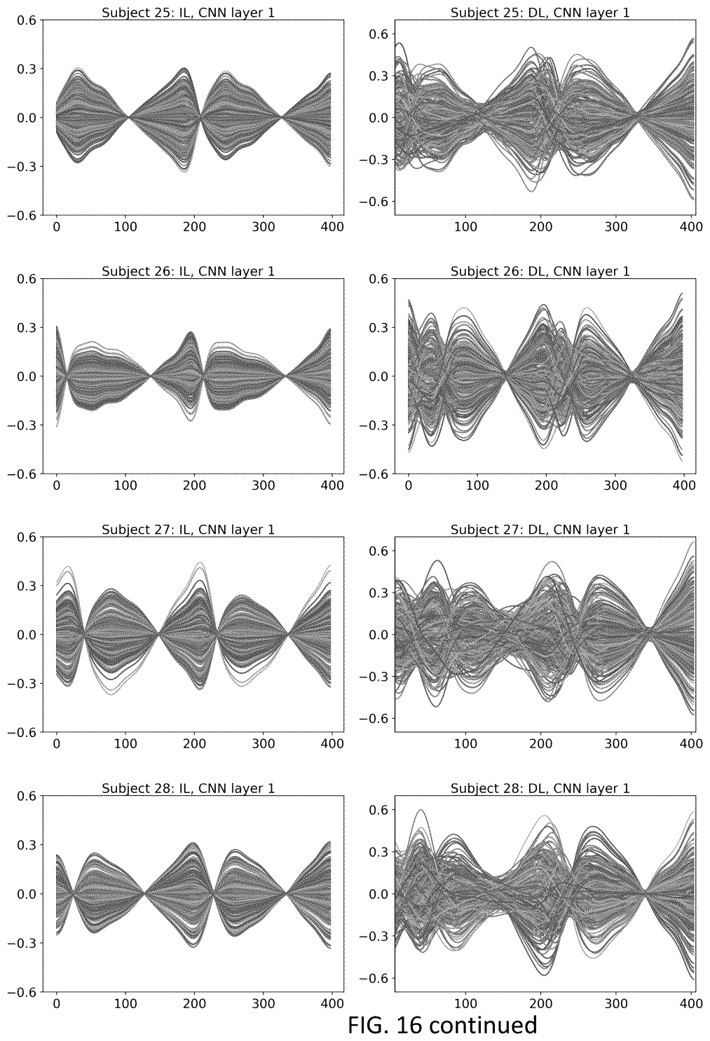

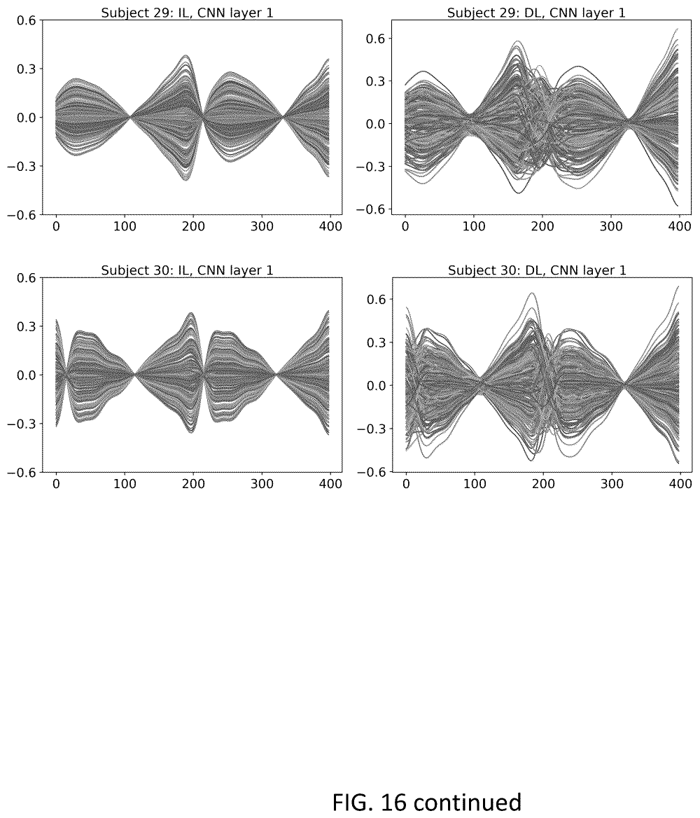

The idea of pairing data of adjacent rounds is based on the assumption that there exists a correlation between blood glucose level variation and the PPG signal variation. Although the correlation between rounds could also be learned from traditionally separated single input (IL), the model training could be quite inefficient and may often result in overfitting. In this work, we set out to design a more efficient way to enhance model learning with limited data through the comparison of two rounds of PPG signals. Here we demonstrate the result of a signal segment passing through a specific CNN filter, to illustrate the possible reason of superior performance in DL models 10 over IL models 8. FIGS. 1d and 1f show a typical example (a real case in round 7 of subject 1) the major difference in the first convolutional layer between 1-channel and 2-channel inputs. With simply 1-channel input that the vector convolves to a specific filter (filter length=3), the corresponding output generates a linear combination of three adjacent input vector elements for each output data point, which is seen as a reverse pattern in our example (FIG. 1c). On the other hand, for 2-channel input vectors, they convolve separately and then are superimposed together to form the output. This step creates more possible variations of operations to the original signal, including the one shown in FIG. 1e. As a result, unlike the 1-channel input, which reveals similar pattens in each pulse, 2-channel input leads to extra and non-uniform features than the 1-channel input, i.e., more complicated local peaks and valleys in the waveforms of feature patterns, and the feature patterns of different pulses might be quite different. This might make the model easier to establish the correspondence between the features and the predicted BGL, and also to avoid overfitting in a long period of training. More evidence of model with 2-channel input results in a more complicated structure of features than with 1-channel input is illustrated in FIGS. 1d and 1f, and in FIG. 16 for more of our tested subjects, which show the output of the first CNN layer of all 256 filters for both cases. In these examples, each 1.6 second segment contains about two pulses of PPG signal. For the case of similarly repeated two-pulse morphology in the segment, it is clear that only one pattern with positive and negative scaling comprises all the output for the model with 1-channel input; while more variations exist in the results of the model with 2-channel input. As a result, with the impose of our domain knowledge (Eq.1, FIG. 1b), 2-channel input in DL has more potential to learn complex tasks, including the relatively weak correlation between PPG signals and BGLs. For the details of composing 2-channel input for our model, please refer to the section Method.

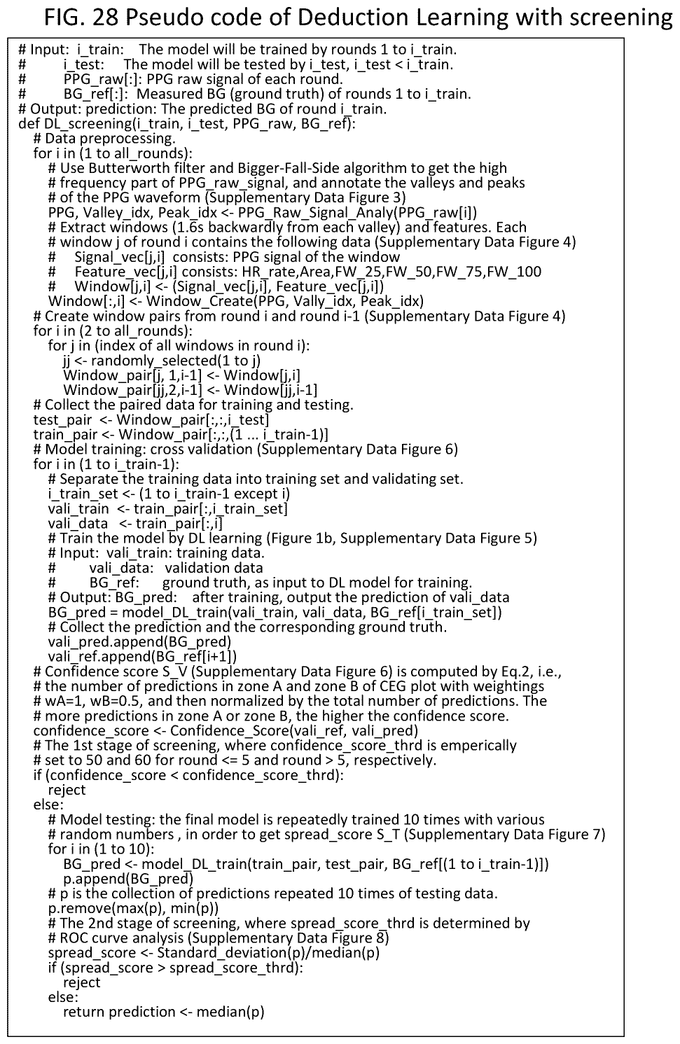

Considering clinical application, although DL model 10 was demonstrated to significantly outperformed IL model 8 in prediction accuracy (see section Results), a screening algorithm can be used to further exclude outliers arising from abnormal measurement of PPG signals. Our implementation of screening algorithm is illustrated in FIGS. 7A and 7B, together with the pseudo code presented FIG. 28, in which two stages of screening process are implemented based on the test of validation confidence score S_{V} and the test spread score S_{T}, respectively (see section Method for the definitions of S_{V} and S_{T}). In the first stage, S_{V} is used to examine quality of the model built from the training data of all the preceding rounds. The model quality is highly sensitive to the amount and quality of the training data. If the model cannot pass the screening of the first stage, it usually means that more rounds of PPG measurements, together with the corresponding reference BGL 230 from finger-pricks, are needed for model building. In the second stage of screening, Sr calculated from the model prediction of the testing data is checked to filter out possible outliers. This usually due to an inferior measurement of PPG signal 200, and redo the measurement more rigorously is usually necessary.

The threshold values of S_{V} and S_{T} for pass/reject decision of the built model and predictions were determined by the empirical tests and the receiver operating characteristic (ROC) curve³⁶ (see FIG. 8), respectively. For a given model with accepted quality (i.e., the first screening stage is passed), ROC curve provides a systematic way to search for an optimal threshold value of S_{T} to filter out abnormal predictions. Ideally, the optimal threshold should be found near the convex point close to the upper-left corner of ROC curve plot. If the threshold is shifted along the curve to the upper right side, the condition is less stringent and more samples are accepted.

If it is shifted towards the lower left side, the condition is stricter and fewer samples are included. FIG. 8 shows the ROC curve calculated from all subjects after eight rounds of measurements for our models. It is interesting to compare the effect of S_{T} screening on both DL and IL, which reveals the superiority of DL over IL more clearly. The optimal threshold values of S_{T} for both cases are near 0.07, with the corresponding locations in ROC curve illustrated by the red dots. With this threshold value, the true positive rates of DL and IL are 78.3% and 50.8% (FIG. 8a), and the reject ratios are 31.1% and 56.5% (FIG. 8b), respectively. Furthermore, the ROC curve of IL is closer to the diagonal line, which indicates that the true positive rate and the false positive rate are simultaneously growing with respect to relaxing threshold. In this case, optimization of the threshold value would not improve the pass/reject accuracy significantly. On the contrary, the ROC curve of DL shows a promising shape above the diagonal line, which indicates the spread score S_{T} is more effective to distinguish the normal and abnormal test predictions in the DL model. Therefore, we conclude that pairing of adjacent rounds of data in DL helps screening task and achieves an overall better performance.

### Results

In this work, our codes were developed in Python 3.6.6, with tensorflow 1.11.0, keras 2.2.4, on a platform of CUDA driver/runtime versions 10.1/9.0, and cuDNN 7.3.1.20. All the model training and testing were performed on an ASUS ESC8000 server with dual Intel Xeon Silver 4114 CPUs, 6 GPU cards of GTX 1080Ti (11 GB GPU on board memory), and 256 GB host memory. The training of the CNN architecture (FIG. 3) was performed in GPUs, and the required training time and GPU resources are summarized in Table 4. The column “training rounds” means training from round 1 to the listed rounds. Note that our architecture of both IL and DL are accumulated training, for example, training up to round 4 of DL involves three DC trainings of pairs (S₁,S₂), (S₂,S₃), and (S₃,S₄) (see FIG. 1). To speed up the trainings as much as possible, in each case we performed all the involved NN (for IL) or DC (for DL) trainings parallelly. Thus, for trainings up to more rounds, the required GPU resources grew roughly linearly.

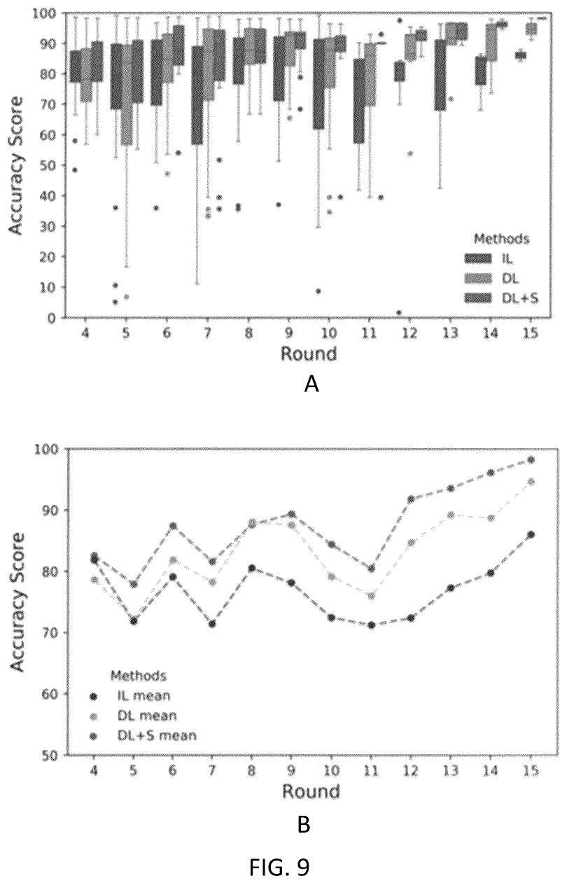

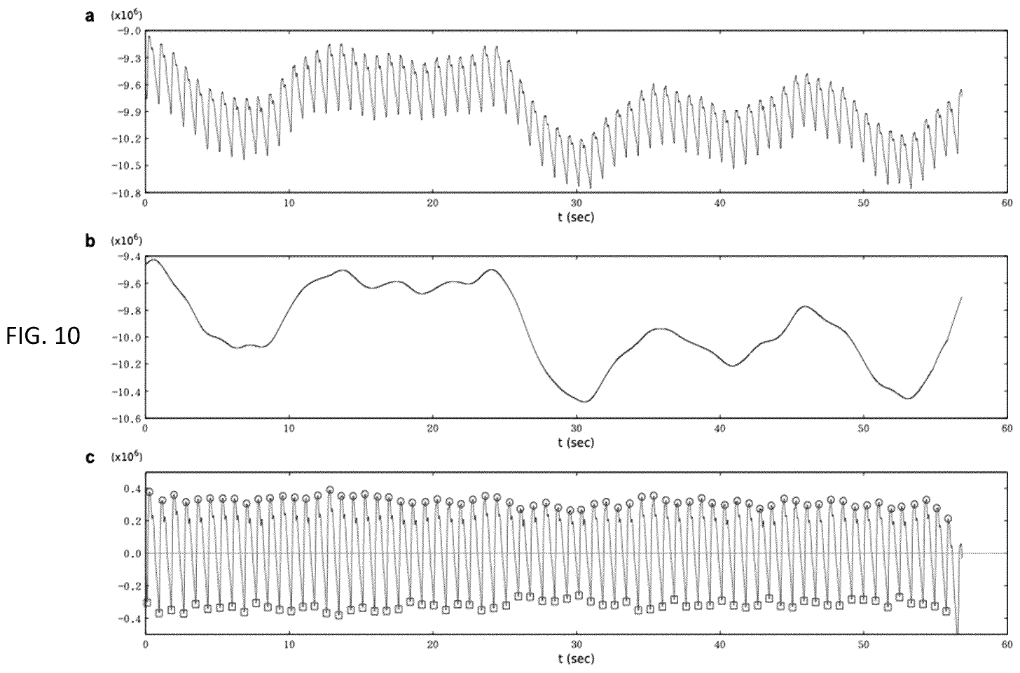

To present the prediction results in a timeline scenario, in FIGS. 9a and 9b we show the average and variation of predicting accuracy score of all subjects for every test round since round 4. In this Example, the accuracy score is defined in Eq.4, which is the relative difference between the predicted BGL 300 and the ground truth BGref 230. Here (and also all the following results unless described specifically), the result of round i represents the prediction of PPG signal samples of paired (for DL and DL+S) data from round i and round i−1, and non-paired (for IL) data from round i, respectively, by the corresponding models built with the training data collected in the preceded rounds from round 1 to round i−1. Evidently, marked improvement of the mean accuracy scores is found between DL (orange line) and IL (blue line). Furthermore, the variation of prediction accuracy of IL is much larger than that of DL. This result strongly suggests that DL model 10 outperforms IL model 8 both in prediction accuracy and stability. This enhancement is due to the influence of data pairing. In addition, with the screening enabled, the mean accuracy score of DL+S (DL with screening, green line) is further improved to more than 90 after round 11. This demonstrates that our screening mechanism works prominently in filtering out the bad predictions.

Due to the reduced number of subjects in the later rounds (see Table 2), the averaged accuracy of each round may not be representative to reveal the true statistics. In order to disclose the real trend of prediction accuracy in the long term, in

Table 5, the overall performance of three models, IL 8, DL 10, and DL+S 20 are presented in groups of rounds 4˜7, 8˜11, 12˜15, and the total (4˜15). More detailed data can be found in Table 3. Comparing IL 8 and DL 10, it is clear that, unlike DL 10, both the prediction accuracy and Pearson correlation coefficient do not improve with increasing rounds of training data. There are fluctuations, which lead to an overall almost flattened trend in accuracy score but actually decreasing correlation with more accumulated training data. This seems to be a common phenomenon of the traditional IL 8, that long-term NIBG prediction from PPG signals may gradually fail, and it seems to be helpless with more training data added. On the other hand, DL 10 has significant improvement in prediction with more and more training data accumulated. This exhibits a remarkable difference between IL 8 and DL 10. Furthermore, enabling screening to separate out the bad predictions, both the accuracy and the correlation gain more improvement of >0.06 and significantly >0.17 over DL, respectively, in the group of rounds 12˜15.

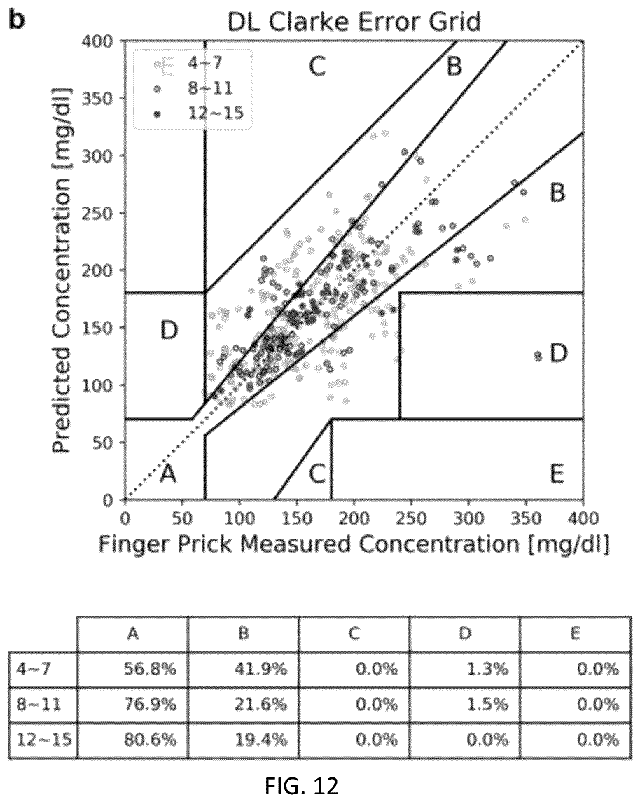

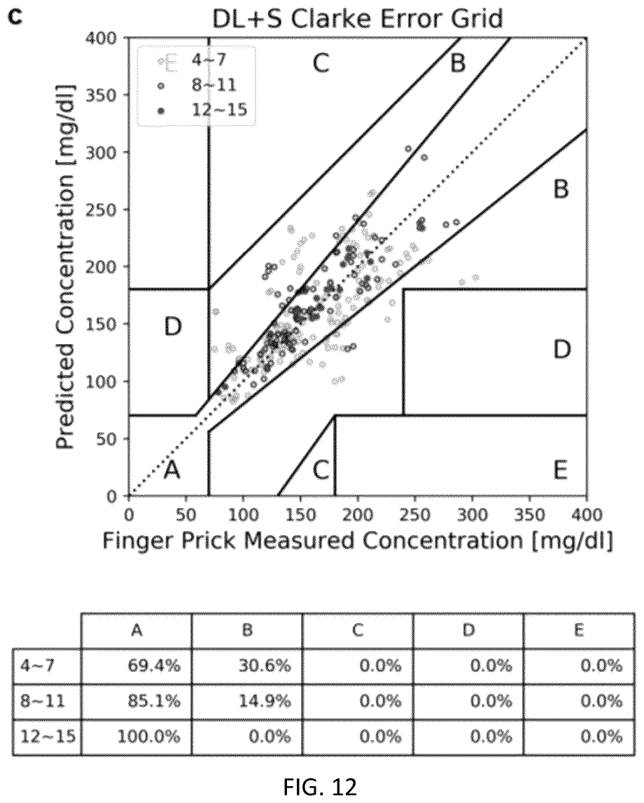

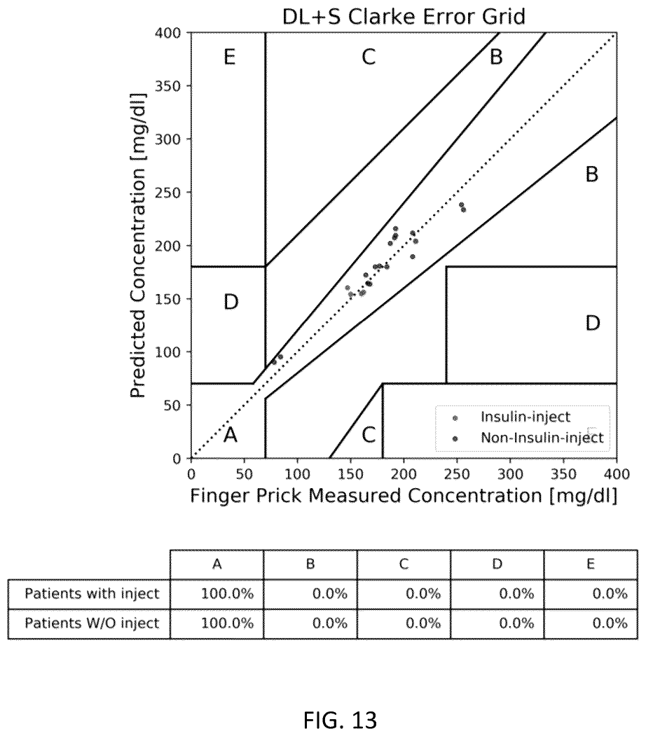

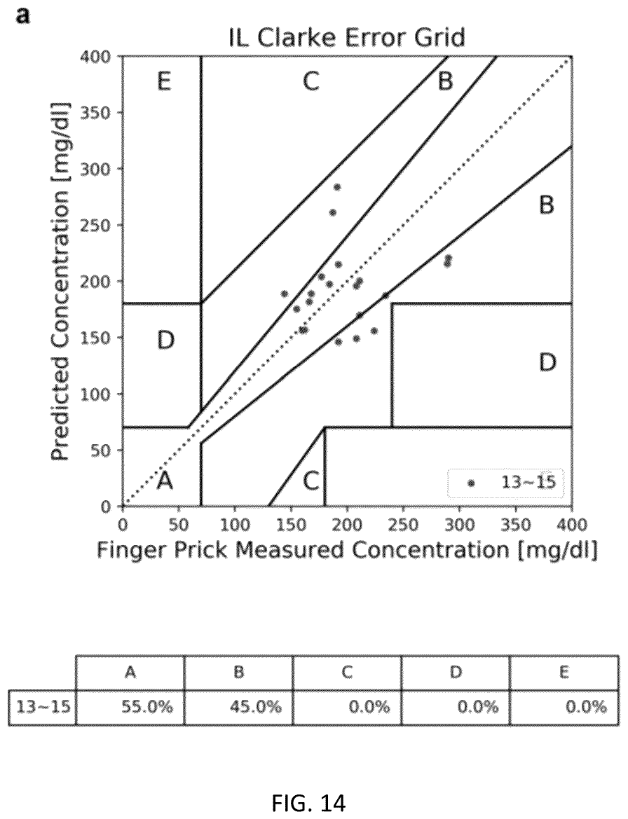

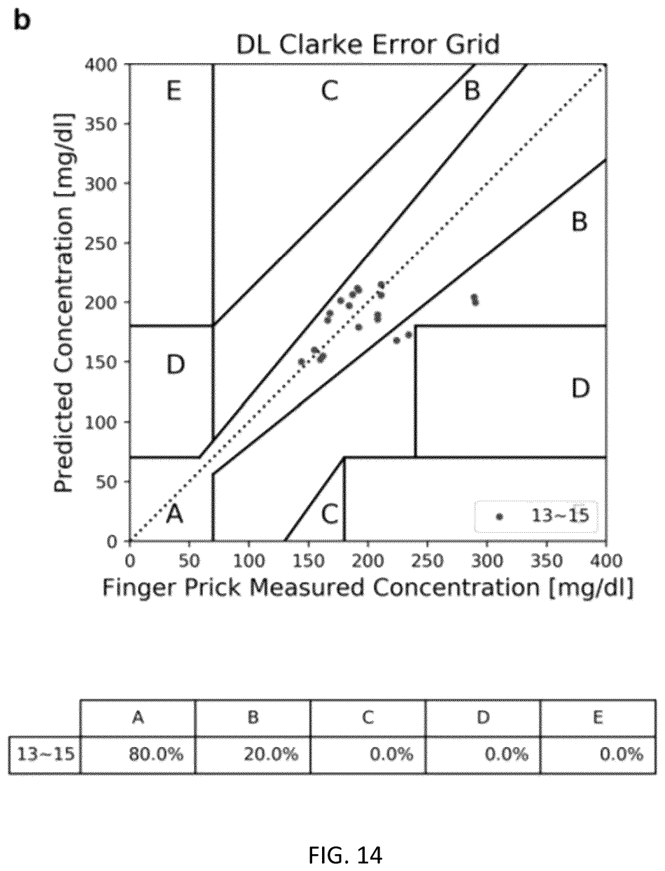

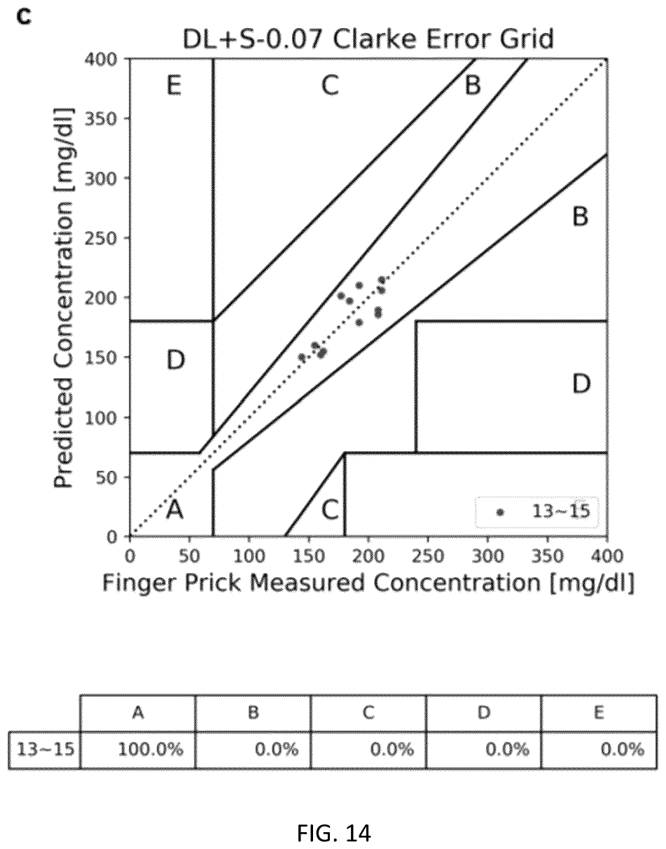

The difference between the predicted BGL 300 versus the reference BGL 230 can be more clearly visualized by Clarke Error Grid (CEG) plots 37. Comparing IL 8 and DL 10, as shown in FIGS. 12a and 12b, the predicted data points in zone A are significantly increased in rounds 8˜11 and rounds 12˜15 of DL, about 19% and 20% improvements over IL in predictions in zone A of CEG, except the particular outlier point (blue, at round 10) in zone D of IL 8 and DL 10. Examining this outlier more closely, it possesses a high reference BGL (>350 mg/dl), higher than BGLs in the preceded rounds utilized to train the model. Nevertheless, with screening enabled (FIG. 3c), this outlier is removed. As a result, with DL+S 20, it not only gives enhanced prediction accuracy, but also delivers a more robust result without the erroneous and misleading predictions.

The influence of insulin injection on the accuracy of BGL prediction was also investigated. As illustrated in FIG. 4, stratification of DM patients into with/without insulin injection groups illustrates a similar pattern in the CEG plot. This demonstrates the uniformity of DL+S model prediction on DM patients regardless of medical treatment.

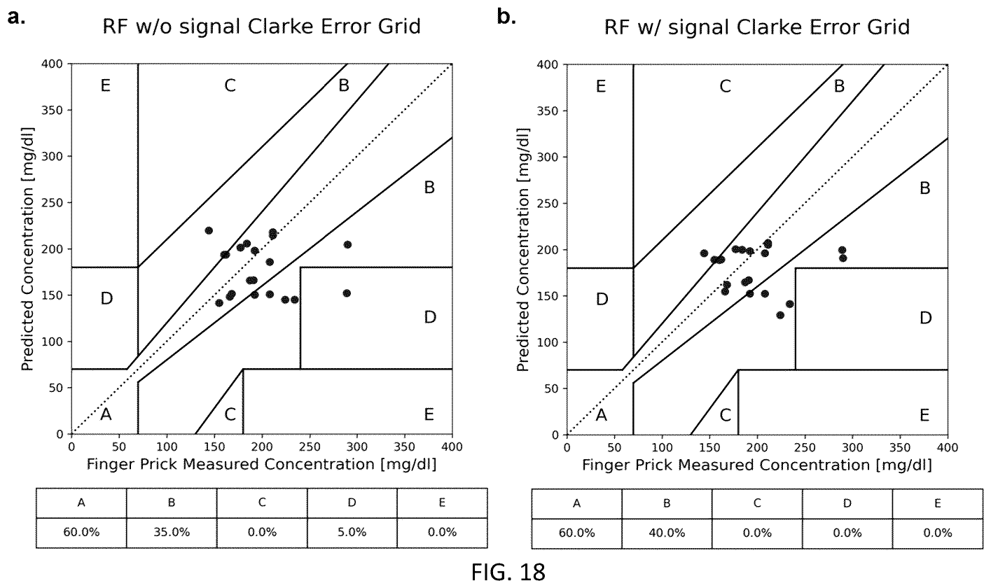

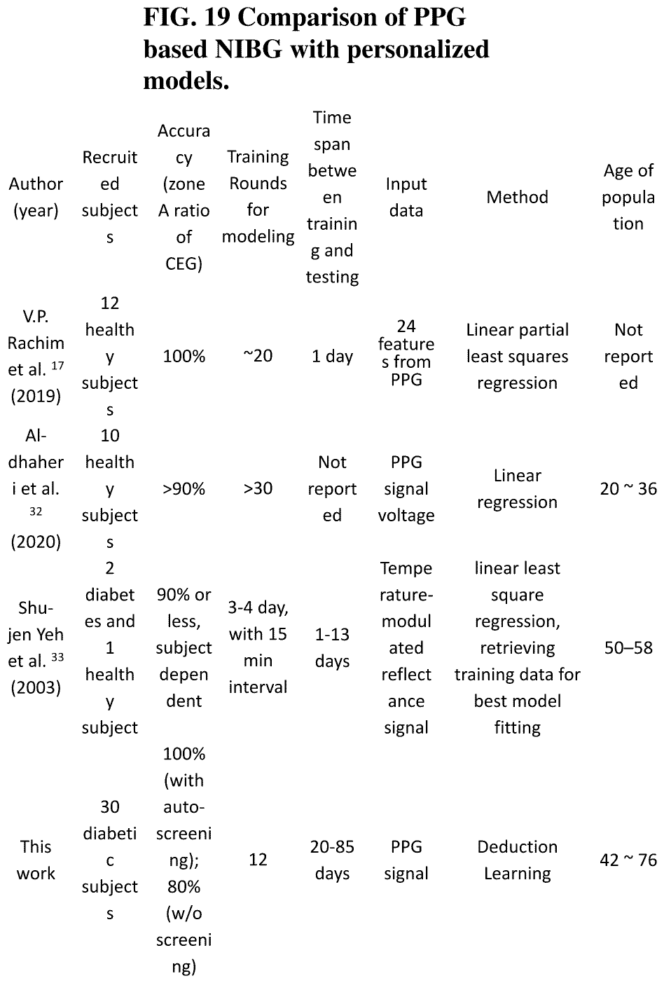

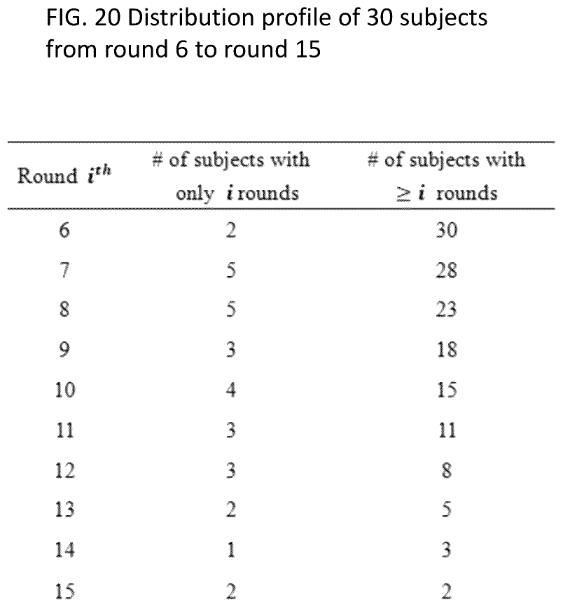

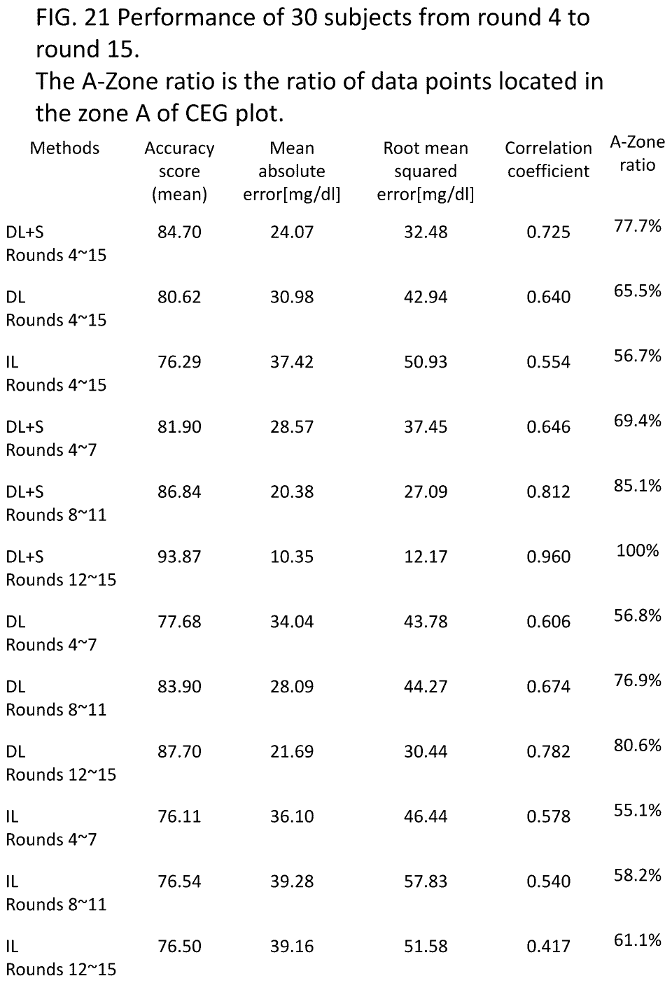

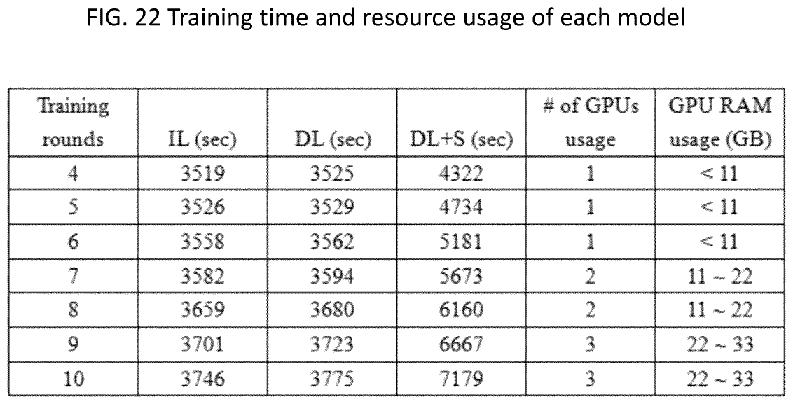

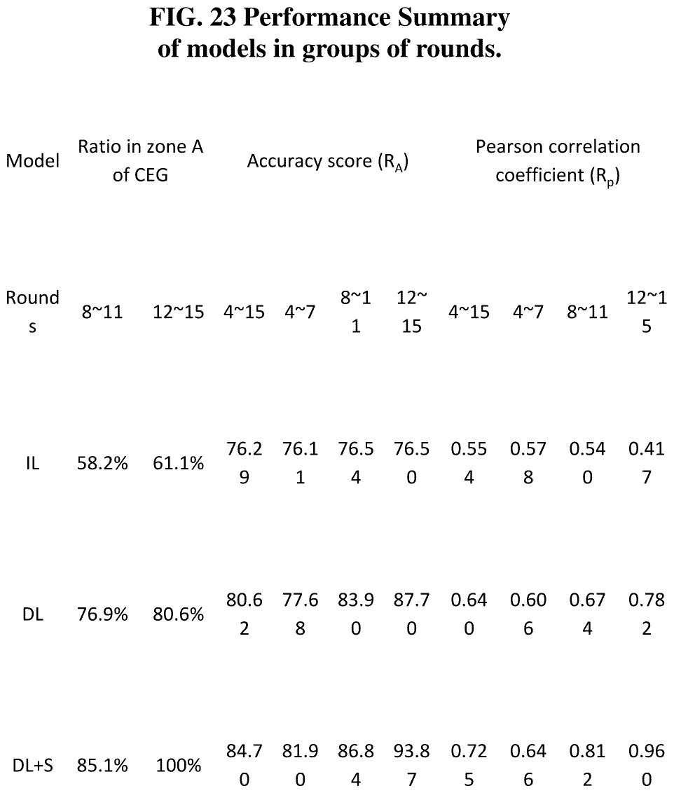

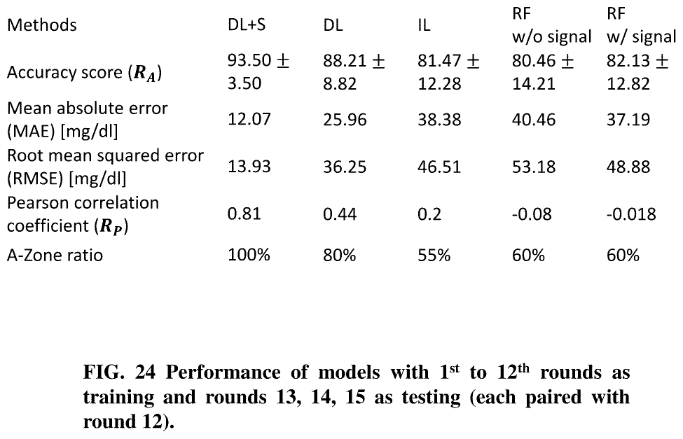

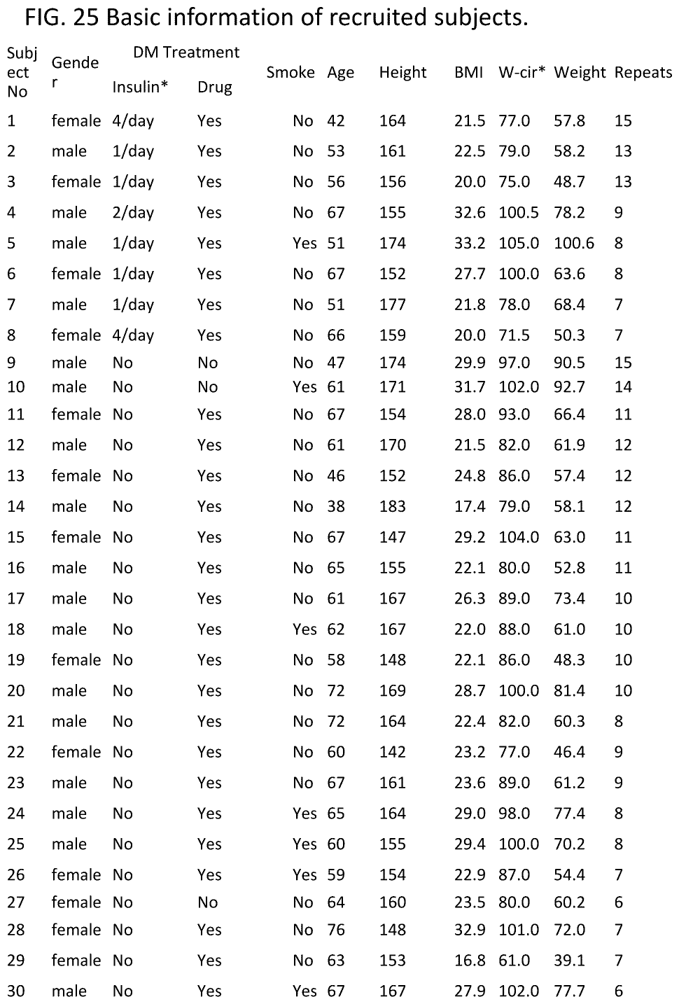

Finally, we test a scenario of a real application. For BGL estimation based on a personalized model, the first step is training the model with enough personal data, together with the corresponding real BGL measurements from finger-pricks, and then uses the model for forthcoming predictions. A clinically useful model should be able to preserve prediction quality for a reasonably long period, without adding more training data to regulate the model from deviations. In this test, the models were built with a dozen rounds of training data (rounds 1˜12), and then were tested in rounds 13˜15. The data pairing for running this test is data of round 12 paired with data of rounds 13˜15 to predict BGLs for rounds 13˜15, respectively. The prediction results are presented in Table 6, FIG. 15, and FIG. 18. Here, for completeness, we also incorporate results of our preliminary tests of Random Forest (RF) model, to illustrate the general property of performance difference between IL 8 and DL 10.

In our preliminary tests, the RF models 6, which are also regarded as the framework of IL, were trained with 6 morphological features data only (labeled as “RF w/o signal”), and with both 6 morphological features and PPG signals data (labeled as “RF w/signal”), of rounds 1˜12, respectively. For IL 8 and DL (+S) 10, 20, we used both 6 morphological features 240 and PPG signals data for model training (see section Methods, Data Preprocessing, and FIG. 11). But for the RF models 6, in practice usually only distinct features are used as the training data, like the case of “RF w/o signal”. Here for completeness and fair comparison, we also tried the case of “RF w/signal”, i.e., using exactly the same training data as that of IL 8 and DL (+S) 10, 20 for the RF models.

Our prediction result shows the accuracy of DL+S 20 outperformed DL 10, and DL 10 outperformed IL 8 and RF 6. Comparing IL 8 and the two RF 6 results, although they have similar RA, MAE, RMSE, and A-zone ratio, but Rp (Pearson correlation coefficient) of the two RF results is worse than that of IL. This is also apparent in CEG plots. Comparing Supplementary Data FIG. 11 and FIG. 15a, the data points of RF results tend to lie flat instead of lying along the diagonal line, which means that it is more difficult for RF to figure out the correlation of input data and BGL, and thus produced worse predictions. Next, comparing IL 8 and DL (+S) 10, 20, almost half of predicted data points reside in zone B of CEG plot for IL 8, and for DL and DL+S 20, the predicted data points in zone B are reduced to 20%, and completely removed with the help of screening, respectively. All the other metrics 10 also show the performance enhancement of DL (+S) 10, 20 over IL 8. In other words, DL 10 gives more promising predictions than IL 8, and DL+S 20 ensures the confidence of predictions in minimal obscurities.

Finally, in order to objectively compare the performance between DL 20 and the models of IL 8 and RF 6 models individually with quantified metrics, the nonparametric Wilcoxon³⁸ paired test of these predicting results was conducted. Among the performance metrics listed in Table 6, only R_{A} has a data population in each model, hence R_{A} was used to perform the paired test. Here we were aiming to compare the direct predictions of DL 10 with IL 8 and RF 6 models specifically, thus only the paired tests of DL 10 with these models were conducted separately. Further, the tests of DL+S 20 with the other models were skipped, because DL+S 20 essentially gave the same predictions as DL 10, except that some predicted data considered as outliers were removed by screening. The Wilcoxon paired test results of R_{A} gives p-value=0.063 for DL 10 versus IL 8, 0.068 for DL 10 versus RF 6 w/o signal, and 0.165 for DL 10 versus RF 6 w/signal, respectively. These p-values were just near significant (0.05) due to some overlaps of their R_{A} distributions (as indicated in standard deviations of R_{A}, see Table 6). But from viewpoint of clinical criteria, such as Rₚ and A-zone ratio, DL 10 significantly overwhelmed IL 8 and RF 6 models by more than 20% improvement. As a result, we conclude that DL (+S) 20 is significantly outperformed IL 8 and RF 6 for non-invasive BGL prediction.

### Conclusion

To solve the problem of inaccurate personalized NIBG (noninvasive blood glucose) prediction due to insufficient amount of training data, a novel prediction method, Deduction Learning (DL), is presented. The DL method involves the use of paired adjacent rounds of finger pulsation Photoplethysmography (PPG) signal recordings as the input to a convolutional-neural-network (CNN) based deep learning model. It reliably predicts fasting blood glucose level (BGL) of diabetes mellitus (DM) patients, either with or without insulin injections. For subjects with 12 rounds of data for model training and tested with rounds 13˜15, DL+S achieved an accuracy score of 93.50, a root mean squared error (RMSE) of 13.93 mg/dl, and a mean absolute error (MAE) of 12.07 mg/dl, in which the improvement in accuracy over the conventional method is more than 12%. The paired t-test on MAE and (R_{A}) of DL with respect to IL also revealed highly significant in predicting power, with p-values smaller than 0.04 and 0.03, respectively. Furthermore, DL+S attended 100% of prediction data in zone A of CEG plot. This significant enhancement might be attributed to more feature patterns arising from CNN process with the pairing mechanism. It emphasized the differentiation between two contiguous PPG records, which is the direct consequence of imposing (Eq.1, FIG. 1b) in our model design to guide the learning, that could be helpful for CNN to find the concealed correlation between the PPG signal and BGL. It also indicates that a very simple and precise noninvasive measurement of BGL is achievable. Given more amount and types of subject-specific data accumulated, it would be promising not only to enhance the prediction quality of DL, but also to explore more possibilities in biomedical and clinical applications.

Moreover, with imposing the rule (Eq.1, FIG. 1b) in DL model design, the guided learning process demonstrated a prominent example of deduction learning implementation. We hope it could also contribute to more innovative ideas of the model design for other data insufficient and realistic applications with machine learning.

### REFERENCES

### Each Reference is Incorporated in Each of their Entirety

1 Silver, D. et al. Mastering the game of Go with deep neural networks and tree search. Nature 529, 484-489, doi: 10.1038/nature16961 (2016).
2 DeFronzo, R. A., Ferrannini, E., Zimmet, P. & Alberti, G. International textbook of diabetes mellitus. (John Wiley & Sons, 2015).
3 Hall, A. P. & Davies, M. J. Assessment and management of diabetes mellitus. The Foundation Years 4, 224-229 (2008).
4 Sarkar, K., Ahmad, D., Singha, S. K. & Ahmad, M. in 2018 21st International Conference of Computer and Information Technology (ICCIT) 1-5 (IEEE, Dhaka, Bangladesh, 2018).
5 Mekonnen, B. K., Yang, W., Hsieh, T. H., Liaw, S. K. & Yang, F. L. Accurate prediction of glucose concentration and identification of major contributing features from hardly distinguishable near-infrared spectroscopy. Biomedical Signal Processing and Control 59, 101923, doi: 10.1016/j.bspc.2020.101923 (2020).
6 Maier, J. S., Walker, S. A., Fantini, S., Franceschini, M. A. & Gratton, E. Possible Correlation between Blood-Glucose Concentration and the Reduced Scattering Coefficient of Tissues in the near-Infrared. Optics Letters 19, 2062-2064, doi: 10.1364/01.19.002062 (1994).
7 Tamada, J. A. et al. Noninvasive glucose monitoring: comprehensive clinical results. Cygnus Research Team. JAMA 282, 1839-1844, doi: 10.1001/jama.282.19.1839 (1999).
8 Klonoff, D. C. Noninvasive blood glucose monitoring. Diabetes Care 20, 433-437, doi: 10.2337/diacare.20.3.433 (1997).
9 Larin, K. V., Eledrisi, M. S., Motamedi, M. & Esenaliev, R. O. Noninvasive blood glucose monitoring with optical coherence tomography: a pilot study in human subjects. Diabetes Care 25, 2263-2267, doi: 10.2337/diacare.25.12.2263 (2002).
10 Yadav, J., Rani, A., Singh, V. & Murari, B. M. Prospects and limitations of non-invasive blood glucose monitoring using near-infrared spectroscopy. Biomedical Signal Processing and Control 18, 214-227, doi: 10.1016/j.bspc.2015.01.005 (2015).
11 Chen, Y. et al. Skin-like biosensor system via electrochemical channels for noninvasive blood glucose monitoring. Sci Adv 3, e1701629, doi: 10.1126/sciadv.1701629 (2017).
12 Abd Salam, N. A. B., bin Mohd Saad, W. H., Manap, Z. B. & Salehuddin, F. The evolution of non-invasive blood glucose monitoring system for personal application. Journal of Telecommunication, Electronic and Computer Engineering (JTEC) 8, 59-65 (2016).
13 Freer, B. & Venkataraman, J. in 2010 IEEE Antennas and Propagation Society International Symposium. 1-4 (IEEE).
14 Blank, T. B. et al. in Optical Diagnostics and Sensing of Biological Fluids and Glucose and Cholesterol Monitoring II. 1-10 (International Society for Optics and Photonics, 2002).
15 Paul, B., Manuel, M. P. & Alex, Z. C. in 2012 1st International Symposium on Physics and Technology of Sensors (ISPTS-1). 43-46.
16 Ramasahayam, S., Arora, L., Chowdhury, S. R. & Anumukonda, M. in 2015 9th International Conference on Sensing Technology (ICST). 22-27.
17 Rachim, V. P. & Chung, W. Y. Wearable-band type visible-near infrared optical biosensor for non-invasive blood glucose monitoring. Sensors and Actuators B-Chemical 286, 173-180, doi: 10.1016/j.snb.2019.01.121 (2019).
18 Maruo, K. et al. New methodology to obtain a calibration model for noninvasive near-infrared blood glucose monitoring. Appl Spectrosc 60, 441-449, doi: 10.1366/000370206776593780 (2006).
19 Jain, P., Joshi, A. M. & Mohanty, S. P. iGLU 1.0: An Accurate Non-Invasive Near-Infrared Dual Short Wavelengths Spectroscopy based Glucometer for Smart Healthcare. arXiv preprint arXiv: 1911.04471, doi: 10.1109/MCE.2019.2940855 (2019).
20 Karimipour, H., Shandiz, H. T. & Zahedi, E. Diabetic diagnose test based on PPG signal and identification system. Journal of Biomedical Science and Engineering 2, 465-469 (2009).
21 Monte-Moreno, E. Non-invasive estimate of blood glucose and blood pressure from a photoplethysmograph by means of machine learning techniques. Artif Intell Med 53, 127-138, doi: 10.1016/j.artmed.2011.05.001 (2011).
22 Hina, A., Nadeem, H. & Saadeh, W. in 2019 IEEE International Symposium on Circuits and Systems (ISCAS). 1-5.
23 Turksoy, K. et al. Hypoglycemia Early Alarm Systems Based On Multivariable Models. Ind Eng Chem Res 52, 12329-12336, doi: 10.1021/ie3034015 (2013).
24 Wold, S., Sjostrom, M. & Eriksson, L. PLS-regression: a basic tool of chemometrics. Chemometrics and Intelligent Laboratory Systems 58, 109-130, doi: Doi 10.1016/S0169-7439 (01) 00155-1 (2001).
25 Bunescu, R., Struble, N., Marling, C., Shubrook, J. & Schwartz, F. in 2013 12th International Conference on Machine Learning and Applications. 135-140 (IEEE).
26 Georga, E. I., Protopappas, V. C., Polyzos, D. & Fotiadis, D. I. in 2012 Annual International Conference of the IEEE Engineering in Medicine and Biology Society. 2889-2892 (IEEE).
27 Altman, N. S. An Introduction to Kernel and Nearest-Neighbor Nonparametric Regression. Am Stat 46, 175-185, doi: Doi 10.2307/2685209 (1992).
28 Zhang, G. B. et al. A Noninvasive Blood Glucose Monitoring System Based on Smartphone PPG Signal Processing and Machine Learning. Ieee Transactions on Industrial Informatics 16, 7209-7218, doi: 10.1109/Tii.2020.2975222 (2020).
29 Tomczak, J. M. in Advances in Systems Science. (eds Jerzy Świątek & Jakub M. Tomczak) 98-108 (Springer International Publishing).
30 Eberly, L. E. Multiple linear regression. Methods Mol Biol 404, 165-187, doi: 10.1007/978-1-59745-530-5_9 (2007).
31 Yadav, J., Rani, A., Singh, V. & Murari, B. M. Investigations on Multisensor-Based Noninvasive Blood Glucose Measurement System. Journal of Medical Devices-Transactions of the Asme 11, doi: 10.1115/1.4036580 (2017).
32 Al-Dhaheri, M. A., Mekkakia-Maaza, N.-E., Mouhadjer, H. & Lakhdari, A. Noninvasive blood glucose monitoring system based on near-infrared method. International Journal of Electrical & Computer Engineering (2088-8708) 10 (2020).
33 Yeh, S. J., Hanna, C. F. & Khalil, O. S. Monitoring blood glucose changes in cutaneous tissue by temperature-modulated localized reflectance measurements. Clin Chem 49, 924-934, doi: 10.1373/49.6.924 (2003).
34 Davies, M. J. et al. Management of Hyperglycemia in Type 2 Diabetes, 2018. A Consensus Report by the American Diabetes Association (ADA) and the European Association for the Study of Diabetes (EASD). Diabetes Care 41, 2669-2701, doi: 10.2337/dci18-0033 (2018).
35 Mitchell, T. M. in Machine learning McGraw-Hill International Editions Computer Science Series Ch. 11, (McGraw-Hill, 1997).
36 Hajian-Tilaki, K. Receiver Operating Characteristic (ROC) Curve Analysis for Medical Diagnostic Test Evaluation. Caspian J Intern Med 4, 627-635 (2013).
37 Clarke, W. L., Cox, D., Gonder-Frederick, L. A., Carter, W. & Pohl, S. L. Evaluating clinical accuracy of systems for self-monitoring of blood glucose. Diabetes Care 10, 622-628, doi: 10.2337/diacare.10.5.622 (1987).
38 Conover, W. J. Practical Nonparametric Statistics. 3 edn, 350 (John Wiley & Sons, Inc., 1999).
39 Chu, J., Yang, W.-T., Hsieh, T.-H. & Yang, F.-L. One-Minute Finger Pulsation Measurement for Diabetes Rapid Screening with 1.3% to 13% False-Negative Prediction Rate. Biomedical Statistics and Informatics 6, 8, doi: 10.11648/j.bsi.20210601.12 (2021).
40 Butterworth, S. On the Theory Filter Amplifier S Butterworth. Experimental Wireless and the Wireless Engineer 7, 536-541 (1930).
41 Navakatikyan, M. A., Barrett, C. J., Head, G. A., Ricketts, J. H. & Malpas, S. C. A real-time algorithm for the quantification of blood pressure waveforms. IEEE Trans Biomed Eng 49, 662-670, doi: 10.1109/TBME.2002.1010849 (2002).

What is claimed is:

1. A non-invasive blood glucose prediction model that predicts blood glucose level of a subject based on deduction learning comprising a differential cell, a first input, a second input, a reference blood glucose level input and a predicted blood glucose level output wherein the first input is based upon a first cardiovascular data collected from the subject at a first round of data collection;
the second input is based upon a second cardiovascular data collected from the subject at a second round of data collection;
the reference blood glucose level input is collected from the subject at the first round of data collection;
the differential cell is configured to calculate the predicted blood glucose level of the subject at the time of the second round of data collection based upon the first input, the second input, the reference blood glucose level and correlation between differences between the two inputs and differences between the reference blood glucose level input and the predicted blood glucose level; and
the correlation is learned by the model using deduction learning.

2. The model of claim 1, wherein the first cardiovascular data comprises a first photoplethysmography (PPG) signal collected from the subject at the first round of data collection and the second cardiovascular data comprises a second PPG signal collected from the subject at the second round of data collection.

3. The model of claim 1, wherein the first cardiovascular data, second cardiovascular data and reference blood glucose level are collected from the subject in a fasting state.

4. The model of claim 1, wherein the first input comprises a first set of extracted features extracted from the first cardiovascular signal and the second input comprises a second set of extracted features extracted from the second cardiovascular signal.

5. The model of claim 4, wherein the extracted features comprises heart rate, area under the curve of the waveform, full width, width at 25% of maximum peak amplitude, width at 50% of maximum peak amplitude and/or width at 75% of maximum peak amplitude.

6. The model of claim 1, wherein the reference blood glucose level is obtained using conventional finger prick method.

7. The model of claim 1, wherein the first input comprises one or more signal windows wherein each signal window comprises a digitized segment of the first cardiovascular signal; and
wherein the second input comprises one or more signal windows wherein each signal window comprises a digitized segment of the second cardiovascular signal.

8. The model of claim 7, wherein each segment comprises PPG signal lasting for about 10 second to about 5 minutes such as about 10 seconds, about 30 seconds, about 1 minute, about 1.5 minutes, about 2 minutes, about 2.5 minutes, about 3 minutes, about 3.5 minutes about 4 minutes about 4.5 minutes or about 5 minutes.

9. The model of claim 1, wherein the first round of data collection and the second round of data collection are separated in time by about 1 day, about 2 days, about 5 days about 10 days, about 20 days, about 30 days, about 1.5 months, about 2 months, about 3 months, about 4, months, about 5 months or about 6 months.

10. The model of claim 1, wherein the differential cell comprises a deep neural network configured to learn the correlation between differences between the first input and the second input and differences in the blood glucose levels corresponding to each of the two inputs.

11. The model of claim 1, wherein the differential cell comprises a convolution neural network comprising one or more convolution layers configured to learn the correlation between differences in the first input and the second input and differences in the blood glucose levels corresponding to each of the two inputs.

12. The model of claim 11, wherein the convolution layer comprises one or more 1D convolution layer, batch normalization module, activation module and/or maxpooling module.

13. The model of claim 1, wherein the model is configured to learn the correlation between differences in PPG signal between the first and the second rounds of data collection and differences in blood glucose levels between the first and the second rounds of data collection using deduction learning.

14. The model of claim 1, wherein the model is configured to learn the correlation by deduction learning using a plurality of parallel differential cells, each differential cell with its pair of first historical input and second historical input as well as a pair of first historical reference blood glucose level and a second historical reference blood glucose level wherein the first historical input and the first historical reference blood glucose level are collected from the subject at the first historical round of data collection and the second historical input and the second historical reference blood glucose level are collected from the subject at the second historical round of data collection.

15. The model of claim 14, wherein the deduction learning comprises quantifying differences between the first and second historical inputs, quantifying differences between the first and second historical reference blood glucose levels and determining correlation between the historical input differences and the historical reference blood glucose level differences.

16. The model of claim 15, wherein the correlation minimizes the sum of the absolute values of each historical reference blood glucose level subtracted from the corresponding predicted blood glucose level calculated by each of the differential cell.

17. The model of claim 1, further comprises a screening module configured to improve accuracy of the model.

18. The model of claim 17, wherein the screening module comprises a model quality screening module configured to quantify accuracy of the predicted blood glucose level using historical inputs and historical reference blood glucose levels.

19. The model of claim 18, wherein the model quality screening module quantifies accuracy of predicted blood glucose level based on validation confidence score Sy wherein the validation confidence score S_{V} is calculated from the scattered location of a plurality of the predicted blood glucose level plotted against the corresponding historical reference blood glucose level in a Clark Error Grid (CEG) plot analysis constructed using the standard “leave-one-out” cross-validation procedure.

20. The model of claim 19, wherein validation confidence score is calculated using equation (2) S_{V}=Σ_{i∈U}wᵢCᵢ/Σ_{i∈U}Cᵢ wherein U stands for the union of all zones (from zone A to zone E) of the CEG and wherein wᵢ is the weight of zone i, and Cᵢ is the number of points inside zone i.

21. The model of claim 20, wherein when i=A, w_{A}=1; when i=B, w_{B}=0.5; and when i=C, D, and E, w_{C}=w_{D}=w_{E}=0.

22. The model of claim 17 wherein the screening module further comprises an outlier screening module configured to identify predicted blood glucose level outliers.

23. The model of claim 22 wherein the outlier screening module identifies predicted blood glucose level outliers based on test spread score S parameter calculated from the predicted blood glucose level generated by the model using the corresponding historical inputs and receiver operating characteristic (ROC) curve with removal of maximum and minimum predicted blood glucose level wherein the test spread score is calculated using equation (3)
$${S}_{T} = \sqrt{\frac{{\sum }_{i ∈ n} {( B {G}_{pred}^{( i )} - {\overline{BG}}_{pred} )}^{2}}{N - 1}} / Median ( {BG}_{p r e d} ) ,$$
wherein set n={1, 2, . . . N} contains the repeatedly trained N rounds of PPG and reference blood glucose level data collection and |n|=N−2 due to removal of the maximum and minimum outcomes.

24. The model of claim 23 wherein the test spread score S_{T} parameter is about 0.07 or less.

25. The model of claim 17, wherein the outlier screening module is applied only after the model quality screen module has been applied.

26. The model of claim 14, wherein all the historical inputs and historical reference blood glucose levels used to train the model comes from the subject so that the model is personalized for the subject.

27. A method of generating a predicted blood glucose for a subject using the model of claim 1 comprising the steps of:
collecting the first and the second cardiovascular data as well as the reference blood glucose level from the subject;
creating the first and second input based upon the first and second cardiovascular data;
inputting the first and second inputs into the model;
inputting the reference blood glucose level into the model; and
generating the predicted blood glucose level using the model based on the correlation between differences between the two inputs and differences between the reference blood glucose level input and the predicted blood glucose level.

28. A method of training the model of claim 1 using deduction learning comprising the steps of:
quantifying differences between a first historical input and a second historical input using the differential cell;
quantifying differences between a first historical reference blood glucose level and the second historical reference blood glucose level using the differential cell;
generating a predicted blood glucose level using the differential cell based on the correlation between the differences between the first historical input and the second historical input and differences between the second historical blood glucose level and the predicted blood glucose level; and
adjusting the correlation to minimize absolute value of the difference in value between the second historical reference blood glucose level and the predicted blood glucose level.

29. The method of claim 28, wherein the steps of the method are repeated for a plurality of differential cells using a plurality pairs of historical inputs and a plurality pairs of historical reference blood glucose levels each corresponding to the same round of data collection as each of the historical inputs.

30. The method of claim 29, wherein the correlation minimizes the sum of the absolute values of difference in value between the plurality of historical reference blood glucose level and the corresponding plurality of predicted blood glucose level for each historical round of data collection.

31. The method of claim 28, further comprising the step of screening the model for accuracy.

32. The method of claim 31, further comprising steps of screening accuracy of the model comprising the step of:
plotting each of the plurality of predicted blood glucose level against the corresponding historical reference blood glucose level in a Clark Error Grid (CEG) plot analysis constructed using the standard “leave-one-out” cross-validation procedure, wherein corresponding means the same round of data collection;
calculating validation confidence score using equation (2) S_{V}=Σ_{i∈U}wᵢCᵢ/Σ_{i∈U}Cᵢ wherein U stands for the union of all zones (from zone A to zone E) of the CEG and wherein wᵢ is the weight of zone i, and Cᵢ is the number of points inside zone i; and
eliminating the model if the validation confidence score S_{V} falls below a threshold value.

33. The method of claim 32, wherein when i=A, w_{A}=1; when i=B, w_{B}=0.5; and when i=C, D, and E, w_{C}=w_{D}=w_{E}=0 and threshold value is 50 if number of data collection rounds is <7 and 60 if number of data collection rounds is ≥7.

34. The method of claim 31, further comprising the steps of screening out predicted blood glucose level outliers comprising the steps of
calculating test spread score S_{T} parameter calculated from predicted blood glucose level generated by the model using the historical inputs and receiver operating characteristic (ROC) curve with removal of maximum and minimum BG pred wherein the test spread score is calculated using equation (3)
$${S}_{T} = \sqrt{\frac{{\sum }_{i ∈ n} {( B {G}_{pred}^{( i )} - {\overline{BG}}_{pred} )}^{2}}{N - 1}} / Median ( {BG}_{p r e d} ) ,$$
wherein set n={1, 2, . . . N} contains the repeatedly trained N rounds of PPG and reference blood glucose level collection and |n|=N−2 due to removal of the maximum and minimum outcomes.

35. The method of claim 33 wherein the test spread score S_{T} parameter is about 0.07 or less.

36. The method of claim 31, wherein all the historical inputs and historical reference blood glucose levels used to train the model comes from the subject so that the model is personalized for the subject.
